(19) United States
(12) Patent Application Publication
(10) Pub. No.: US 2024/0002445 A1
(43) Pub. Date: Jan. 4, 2024

(54) TYROSYL-LOCK PEPTIDES

(71) Applicant: The United States of America,as represented by the Secretary,Department of Health and Human Services, Bethesda, MD (US)

(72) Inventors: Barry R. O'Keefe, Frederick, MD (US); Lauren R. Haugh Krumpe, Smithburg, MD (US); Yves Pommier, Bethesda, MD (US); Christophe R. Marchand, Bethesda, MD (US); Ingrid C. Schroeder, Frederick, MD (US); K. Johan Rosengren, Wilston (AU); Brice A.P. Wilson, Frederick, MD (US)

(73) Assignee: The United States of America,as represented by the Secretary,Department of Health and Human Services, Bethesda, MD (US)

(21) Appl. No.: 18/037,379

(22) PCT Filed: Nov. 17, 2021
(86) PCT No.: PCT/US2021/059764

§ 371 Date: May 17, 2023

Related U.S. Application Data
(60) Provisional application No. 63/115,418, filed on Nov. 18, 2020.

(51) Int. Cl. C07K 14/00; C12N 15/63; A61P 35/00; A61K 47/10

(52) U.S. Cl. CPC C07K 14/001; C12N 15/63; A61P 35/00; A61K 47/10

(57) ABSTRACT

Disclosed is a class of knotted cyclic peptides. Related pharmaceutical compositions and methods of using the peptides and methods of synthesizing the peptides are also disclosed.

### CROSS-REFERENCE TO RELATED APPLICATIONS

This patent application claims the benefit of priority to U.S. Provisional Patent Application No. 63/115,418 filed Nov. 18, 2020, which is hereby incorporated by reference in its entirety.

### STATEMENT REGARDING FEDERALLY SPONSORED RESEARCH OR DEVELOPMENT

This invention was made with Government support under project number Z01ZIABC006150 and Z01ZIABC 006161 by the National Institutes of Health, National Cancer Institute. The Government has certain rights in this invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 26,697 Byte ASCII (Text) file named “757881_ST25.txt,” dated Nov. 17, 2021.

### BACKGROUND OF THE INVENTION

Relaxation of supercoiled DNA by topoisomerases is necessary for the normal cell functions of DNA transcription, replication, recombination, and repair. Topoisomerase I (TOP1) mediates both DNA strand break and religation by forming a transient, covalent 3′-phospho-tyrosyl bond with the DNA substrate. This TOP1-DNA cleavage complex is the target of chemotherapeutic TOP1 inhibitors such as the natural product camptothecin. Irinotecan, an analogue of camptothecin, is a widely-used anti-cancer agent that stabilizes the TOP1-DNA cleavage complex, causing irreversible double-strand DNA breaks, eventually leading to the death of replicating cancer cells. Tyrosyl-DNA phosphodiesterase 1 (TDP1) is an enzyme that, upon recognizing stalled TOP1-DNA cleavage complexes, catalyzes the cleavage of the 3′-phopho-tyrosyl bond between DNA and TOP. TDP1 is composed of an as-yet unstructured N-terminal regulatory domain whose function has been reported to be modulated by both phosphorylation and SUMOylation and a C-terminal catalytic domain that utilizes two histidine residues to effect phosphodiester cleavage at Tyr723 of TOP1. After removal of the 3′ adduct, polynucleotide kinase phosphatase prepares the degraded DNA strands for further repair by DNA polymerase 13 and DNA ligase III. The clearance of TOP1-DNA complexes results in escape from TOP1 inhibitor-induced cell death. This activity has led researchers to consider TDP1 a molecular target for the sensitization of replicating cancer cells to camptothecin and related chemotherapeutic agents.

Although these chemotherapeutic agents are effective, they have downsides including negative side effects. Given that cancer is currently a major health concern, there is an urgent need for new TDP1 inhibitors.

### BRIEF SUMMARY OF THE INVENTION

An embodiment of the invention provides knotted cyclic peptides comprising the amino acid sequence of SEQ ID NO: 11

(CX₁X₂XXXCXXXXXXXXXCCXXXXXXSXXLXXXXXXCXXC), wherein X, X₁, and X₂ can be any amino acid provided that at least one of X₁ and X₂ is tyrosine, phenylalanine, or alanine.

An additional embodiment of the invention provide isolated or purified peptides comprising SEQ ID NO: 1, optionally with 1-6 amino acid substitutions or deletions. According to other aspects, there is provided a peptide comprising

(SEQ ID NO: 16)
ZEAFCYSDRFCQNYIGSIPDCCFGRGSYSFELQPPPWECYQC,

optionally with 1-6 amino acid substitutions or deletions; and a peptide comprising

(SEQ ID NO: 30)
GVFCYSDRFCQNPIDN_FDCCFSRGSYSFVPQPTPWDCFQC,

optionally with 1-6 amino acid substitutions or deletions.

Still another embodiment of the invention provides pharmaceutical compositions comprising peptides of an embodiment of the present invention and a pharmaceutically acceptable carrier.

Another embodiment of the invention provides peptides of an embodiment of the present invention, or pharmaceutical compositions of an embodiment of the present invention, for use in treating or preventing cancer.

A further embodiment of the invention provides methods of treating or preventing cancer in a mammal, the method comprising administering to the mammal the peptides of an embodiment of the present invention, or pharmaceutical compositions of an embodiment of the present invention, in an amount effective to treat or prevent cancer in the mammal.

An additional embodiment of the invention provides methods of inhibiting the cleavage of phosphodiester bonds by enzyme Tyrosyl-DNA phosphodiesterase 1 (TDP1) in a mammal, the method comprising administering to the mammal the peptides of an embodiment of the present invention, or pharmaceutical compositions of an embodiment of the present invention, in an amount effective to inhibiting the cleavage of phosphodiester bonds by enzyme TDP1.

Another embodiment of the invention provides nucleic acids encoding the peptides of an embodiment of the present invention, optionally in a vector or a cell.

A further embodiment of the invention provides methods of preparing the peptides of an embodiment of the present invention, by expressing a nucleic acid encoding the peptide in a host cell, optionally wherein the nucleic acid is in a vector.

### BRIEF DESCRIPTION OF THE SEVERAL VIEWS OF THE DRAWING(S)

FIG. 1 is a schematic showing TDP1 processing of 3′-TOP1 DNA adducts and inhibition by an embodiment of the present invention, e.g., recifin A. TOP1 catalyzes single-strand DNA breaks via a transitory, covalent phosphotyrosine linkage involving tyrosine 723 (pTyr723). The cleavage complex is stabilized by the natural product camptothecin, which inhibits DNA religation, trapping TOP1 on the DNA strand, ultimately leading to double strand breaks and cell death. TDP1 removes the 3′-TOP1-pTyr-DNA adducts via a nucleophilic attack on the phosphodiester bond by histidine 263 (H15263) and subsequent hydrolysis by histidine 493 (H15493). After removal of the 3′ adduct, the DNA strand is further enzymatically repaired and re-ligated. Inhibitors of TDP1 catalytic activity, such as recifin A (depicted here by its electrostatic surface potential model) can sensitize cancer cells to TOP1 poisons.

FIG. 2A is a graph showing recifin A inhibition of full-length human TDP1 enzymatic activity. Serial dilutions of purified recifin A were combined with a synthetic 3′-phosphotyrosine capped oligonucleotide DNA substrate and incubated with either full-length recombinant human TDP1 (rhTDP1) or human TDP1 complemented DT40 knockout whole cell extracts (hTDP1 WCE).

FIG. 2B are images of poly-acrylamide gel electrophoresis gels following phosphorimaging of the reactions of FIG. 2A. Activity was calculated as percent of non-inhibited substrate cleavage reaction control.

FIG. 3A is a graph showing disulfide mapping of recifin A, specifically RP-HPLC analysis of partially re-duced and alkylated recifin A. The mixture of native recifin A (3 intact disulfides/3-SS), partially reduced and alkylated isoforms (2 intact disulfides/2-SS, 1 intact disulfide/1-SS) and completely reduced and alkylated recifin A (0-SS) was desalted and separated by RP-HPLC prior to further analysis.

FIG. 3B shows the MS/MS sequencing results for the 2-SS recifin A isoform trypsin fragments established the Cys IV-VI disul-fide linkage (SEQ ID NO: 1). pGlu is pyroglutamic acid; IAA. is iodoacetamide alkylated cysteine; NEM is N-ethylmaleimide alkylated cysteine.

FIG. 3C shows an example of a recifin A disulfide bonding pattern: Cys I-III, Cys II-V, and Cys IV-VI. pGlu is pyroglutamic acid; IAA (SEQ ID NO: 2).

FIG. 4A shows an example of a NMR solution structure of recifin A. The 20 best structures based on MolProbity scores superposed over residues 3-18 and 26-42, emphasising the well-ordered core.

FIG. 4B shows examples of ribbon structures showing the four antiparallel β-strands (I-IV) and the threading of the third β-strand through the ring formed by the three disulfide bonds and β-strands I and IV. The ribbon structure on the right is the ribbon structure on the left rotated 90 degrees.

FIG. 5A shows a ribbon structure of a β-strand threaded Tyr-lock peptide embodiment of the present invention, e.g., recifin A. Recifin A is stabilised by the three disulfide bonds Cys I-III, Cys II-V, and Cys IV-VI, forming a ring together with two of the β-strands, which is penetrated by a third β-strand. The recifin A structure is further stabilised by a central Tyr6 residue locking the structure in place, which is reminiscent of microcin J25.

FIG. 5B shows a ribbon structure of a lasso peptide microcin J25 (PDB ID: 1Q71). Microcin J25, lacks disulfide bonds, but a threaded structure is formed by a cyclisation via an amide-bond between the N-terminal amino-group and the sidechain carboxyl group of Glu8, which creates a circle that wraps around the C-terminal part of the sequence. The threaded structure is locked in place by two aromatic residues, Phe19 and Tyr20, making it sterically impossible for the structure to unravel.

FIG. 5C shows a ribbon structure of a cyclic inhibitory cystine knot peptide kalata B1 (PDB ID: 1NB1). Kalata B1 is the prototypical plant cyclotide, which contains an inhibitory cystine knot motif and a head-to-tail backbone cyclisation. The ICK is formed by three disulfide bonds (Cys I-IV, Cys II-V, Cys III-VI), two of which together with the backbone form a ring that the third disulfide bond is threaded through.

FIG. 5D shows a ribbon structure of a shows a ribbon structure of a β-strand threaded Tyr-lock peptide embodiment of the present invention, e.g., recifin A.

FIG. 5E shows a ribbon structure of a lasso peptide microcin J25 (PDB ID: 1Q71).

FIG. 5F shows a ribbon structure of a cyclic inhibitory cystine knot peptide kalata B1 (PDB ID: 1NB1).

FIG. 6 shows the stabilizing function of Tyr6 (Y6) residue in the overall, Tyr-lock structure of recifin A. Cysll is C11, Tyr14 is Y14, Ser29 is S29, and and Leu32 is L32. Sidechains of residues that pack around Tyr6 are shown with thin lines indicating confirmed inter-residual NOEs.

FIG. 7 is a graph showing the biological activity and specificity of recifin A. Recifin A inhibited full-length TDP1, but not N-terminally truncated TDP1 (Δ147TDP1), enzymatic activity in a concentration-dependent manner with an IC₅₀ of 0.19 μM.

FIG. 8A is a graph showing the steady-state analysis of recifin A modulation of full-length TDP1. Recifin A in-creased both the Km and Vmax kinetic constants of the TDP1 FRET assay24, exhibiting characteristics of both an enzyme inhibitor and activator.

FIG. 8B is a graph showing the effect of recifin A on Δ147TDP1 kinetic parameters. Addition of recifin A did not affect either the Km or Vmax kinetic constants of the Δ147TDP1 FRET assay. As Δ147TDP1 enzyme retained the identical substrate binding and catalytic sites as full-length TDP1, this suggested the allosteric modulation of TDP1, dependent of the N-terminal 147 amino acid residues.

FIG. 9A is a LC-MS analysis of the peptides in bulk-purified Axinella sp. aqueous extract. Total ion chromatogram (TIC), UV absorbance at 280, and the separation gradient are shown.

FIG. 9B is another LC-MS analysis of the peptides in bulk-purified Axinella sp. aqueous extract that were eluted prior to 6 min.

FIG. 9C is another LC-MS analysis of the peptides in bulk-purified Axinella sp. aqueous extract that were eluted prior to 6 min. (60% acetonitrile) showed peptide-like mass-to-charge ratios which deconvoluted to average masses of 4683.87, 4785.89, 4915.95 (recifin), and 5674.47.

FIG. 10A is a LC-MS analysis showing the relative abundance of partially-purified RP-HPLC fraction A from Axinella sp. aqueous peptide extract.

FIG. 10B is a LC-MS analysis showing the relative abundance of partially-purified RP-HPLC fraction B from Axinella sp. aqueous peptide extract.

FIG. 10C is a LC-MS analysis showing the relative abundance of partially-purified RP-HPLC fraction C from Axinella sp. aqueous peptide extract.

FIG. 10D is a LC-MS analysis showing the relative abundance of partially-purified RP-HPLC fraction D from Axinella sp. aqueous peptide extract.

FIG. 11 is graph showing the TDP1 inhibitory activity of the peptide constituents of fractions A-D of Axinella sp. aqueous extract. Fraction A was determined to be the most active and contained the highest abundance of recifin.

FIG. 12A shows a MS analysis of native recifin A. The monoisotopic mass of native recifin was determined to be 4912.9661 Da.

FIG. 12B shows a MS analysis of reduced and alkylated recifin A. The peptide was reduced with 2-mercaptoethanol and alkylated with 4-vinylpyridine (105.06 Da), after which a mass increase of 638.38 Da was observed, indicating the conversion of six cysteine residues to S-pyridylethyl cysteine and three disulfide bonds.

FIG. 13A shows a tandem mass spectra (MS/MS) and automated de novo and amino acid sequencing of recifin A by Edman degradation. Alkylated recifin A tryptic fragment A was subjected to LC-MS and CID MS/MS. PEAKS de novo sequencing software was utilized to interpret the MS/MS spectra. An N-terminal pyro-glutamic acid ion (e) was identified, which prevented Edman degradation analysis (SEQ ID NO: 4).

FIG. 13B shows a tandem mass spectra (MS/MS) and automated de novo and amino acid sequencing of recifin A by Edman degradation. Alkylated recifin A tryptic fragment B was subjected to LC-MS and CID MS/MS. PEAKS de novo sequencing software was utilized to interpret the MS/MS spectra. Fragment B was fully sequenced by Edman degradation. Pyroglutamate aminopeptidase digestion of intact recifin A (reduced and alkylated) afforded Edman degradation sequencing of 35 amino acids, which provided both the order of the tryptic fragments within the molecule and leucine/isoleucine assignments (SEQ ID NO: 5).

FIG. 13C shows a tandem mass spectra (MS/MS) and automated de novo and amino acid sequencing of recifin A by Edman degradation. Alkylated recifin A tryptic fragment C was subjected to LC-MS and CID MS/MS. PEAKS de novo sequencing software was utilized to interpret the MS/MS spectra. Fragment B was fully sequenced by Edman degradation. Pyroglutamate aminopeptidase digestion of intact recifin A (reduced and alkylated) afforded Edman degradation sequencing of 35 amino acids, which provided both the order of the tryptic fragments within the molecule and leucine/isoleucine assignments (SEQ ID NO: 6).

FIG. 14 shows the amino acid sequence of recifin A (SEQ ID NO: 2) and an enzymatic digest map. Reduced and alkylated recifin A was subjected to digestion with various enzymes and sequenced by CID MS/MS to confirm the proposed amino acid sequence. C indicates alkylated, pGlu is pyroglutamic acid. Brackets indicate fragments sequenced by MS/MS. Bolded amino acids in the sequences below indicate the protease recognizes them and digests the polypeptide at that location.

Trypsin:
(SEQ ID NO: 2)
pGluEAFCYSDRFCQNYIGSIPDCCFGRGSYSFELQPPPWECYQC
Glu-C:
(SEQ ID NO: 2)
pGluEAFCYSDRFCQNYIGSIPDCCFGRGSYSFELQPPPWECYQC

Proline endopeptidase:

(SEQ ID NO: 2)
pGluEAFCYSDRFCQNYIGSIPDCCFGRGSYSFELQPPPWECYQC
Chymotrypsin:
(SEQ ID NO: 2)
pGluEAFCYSDRFCQNYIGSIPDCCFGRGSYSFELQPPPWECYQC

FIG. 15A shows a 1D 1H NMR spectra of a ˜2 mg sample of recifin A in 90/10% H₂O/D₂O at 298K acquired on a Bruker AVANCE III equipped with a cryoprobe (ns 32).

FIG. 15B shows secondary Ha chemical shifts compared to random coil values highlighting positive stretches of secondary chemical shifts indicative of β-sheets combined with negative stretches suggesting a-helices.

FIG. 16 is a graph showing the effect of recifin A on TDP1 kinetic parameters.

FIG. 17 shows a Total Correlated Spectroscopy (TOCSY) spectrum of the amide region of recifin A. The amide region shows that the spin systems (numbered) are well dispersed, and it highlights the unusual up-field shift of the NH proton of residue 16 and the Hα proton of Tyr1 1 as well as down-field shift of the Hα proton residue 28.

FIG. 18A shows a ribbon structure illustrating the position of the buried Tyr6.

FIG. 18B shows a schematic illustrating the threading of the third β-strand through the embedded ring formed by the three disulfide bonds. FIG. 18C shows the recifin A sequence; disulfide bond connections are shown with brackets, residues in the ring are at positions 5, 7-11, 21-22, and 39-42 and Tyr at position 6.

FIG. 19 shows a synthetic strategy for recifin A using native chemical ligation of peptide hydrazides.

FIG. 20A shows a superposition of TOCSY spectra of native and synthetic recifin A.

FIG. 20B shows a solution NMR structure of [Phe⁶] recifin showing disulfides.

FIG. 20C shows superposition of [Phe⁶] recifin and native recifin A highlighting the similarities in the Tyr-lock region and hydrogen bonds.

FIG. 21A shows FL-TDP1 FRET Assay results for a reaction progress curve −1 nM, 0.25 μM S, 1XPBS pH 7.4, 80 mM KCl, 1 mM TCEP. FIG. 21B shows FL-TDP1 FRET Assay results for a reaction progress curve −1 nM, 0.25 μM S, 1XPBS pH 7.4, 80 mM KCl.

FIG. 22 shows FL-TDP1 FRET Assay results −1 nM E, 0.25 μM S, T=45 min, 1XPBS pH 7.4, 80 mM KCl.

FIG. 23A shows oxidative folding of recifin A. FIG. 23B shows oxidative folding of [Phe⁶] recifin. HPLC traces of each time point taken for the oxidation of synthetic peptides. Oxidation was performed in 0.1 M ammonium bicarbonate (pH 8.0) with oxidized (0.5 mM) and reduced (2 mM) glutathione at a concentration of 0.125 mg/mL at room temperature. Aliquots were removed at time points 0, 8, and 48 h, quenched with 6 M guanidine hydrochloric acid (pH 3.7). Samples were analyzed by analytical RP-HPLC on a C₁₈ column using a gradient of 5% buffer B for the first 10 min followed by 5-65% B (buffer A: H₂O/0.05% TFA; buffer B: 90% CH₃CN/10% H₂O/0.045% TFA) in 65 min.

FIGS. 24A-24 F show final analytical trace and ESI-MS spectra of oxidized recifin A and analogues.

FIG. 25 shows 1D ¹H Nuclear Magnetic Resonance spectra of recifin A and analogues in 90/10% H₂O/D₂O at 298 K acquired on a Bruker Avance III 900 MHz spectrometer equipped with a cryoprobe. The majority of the purified peptides gave dispersed ¹H NMR spectra with sharp lines, implying that they adopt ordered structures in solution. However, the [Ala⁶] recifin analogue spectra appeared broad and lacked dispersion of the HN signals indicating that the peptide is misfolded. Thus, while substitution of Tyr6 with Phe is well tolerated, incorporating an alanine at position 6 prevents folding of the peptide.

FIG. 26A and 26B are nuclear magnetic resonance scans of recifin A peptides.

FIG. 27 shows aligned sequences of recifin A and analogues with the black line highlighting the disulfide bond connection: Cys I-III, Cys II-V, and Cys IV-VI.

FIG. 28A to 28F show thermal stability (298-333 K) of native recifin A, synthetic recifin A and synthetic recifin A analogues carried out using nuclear magnetic. resonance on a 500 or 700 MHz Bruker Avance III equipped with a cryo probe.

FIG. 29 shows secondary Hα chemical shifts compared to random coil values^{[14]} highlighting positive stretches of secondary chemical shifts indicative of β-sheets combined with negative stretches suggesting α-helices.

FIGS. 30A-30G show ES-MS spectra of recifin A and its analogues hydrazide fragments, as well as the cysteine fragment used for native chemical ligation.

FIG. 31A-31F show ES-MS spectra of ligated recifin A and analogues.

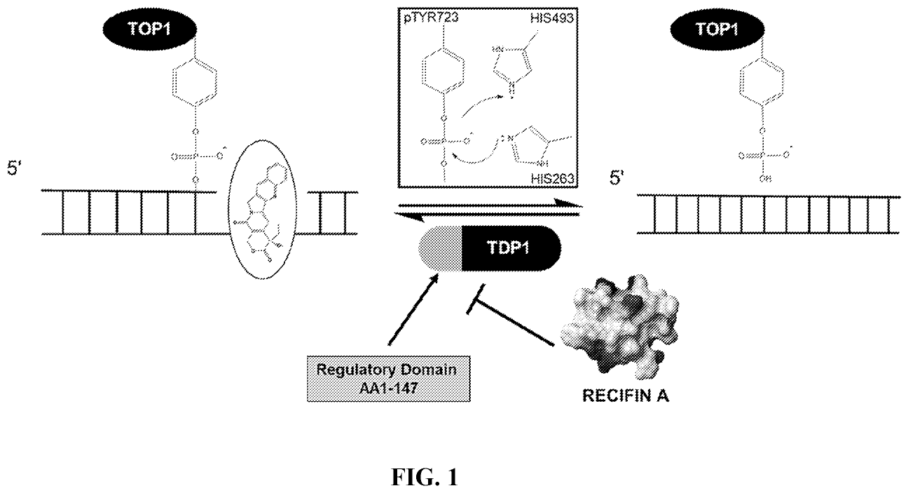

### DETAILED DESCRIPTION OF THE INVENTION

High-throughput screening for inhibitors of TDP1 activity resulted in the discovery of a new class of knotted cyclic peptides from the marine sponge, Axinella sp. Bioassay-guided fractionation of the source extract resulted in the isolation of the active component which was determined to be an unprecedented 42-residue cysteine-rich peptide named recifin A. The native NMR structure revealed a novel fold comprising a four strand anti-parallel β-sheet and two helical turns stabilized by a complex disulfide bond network that creates an embedded ring around one of the strands. The resulting structure, called herein a “Tyr-lock peptide” is stabilized by a tyrosine residue locked into three-dimensional space.

Recifin A inhibited the cleavage of phosphodiester bonds by TDP1 in a Förster resonance energy transfer assay (FRET) with a IC₅₀ of 190 nM. Enzyme kinetics studies revealed that recifin A can specifically modulate the enzymatic activity of full-length TDP1 while not affecting the activity of a truncated catalytic domain of TDP1 lacking the N-terminal regulatory domain (Δ1-147), suggesting an allosteric binding site for recifin A on the regulatory domain of TDP1. This is a previously unknown mechanism of TDP1 inhibition that could be used for anticancer applications.

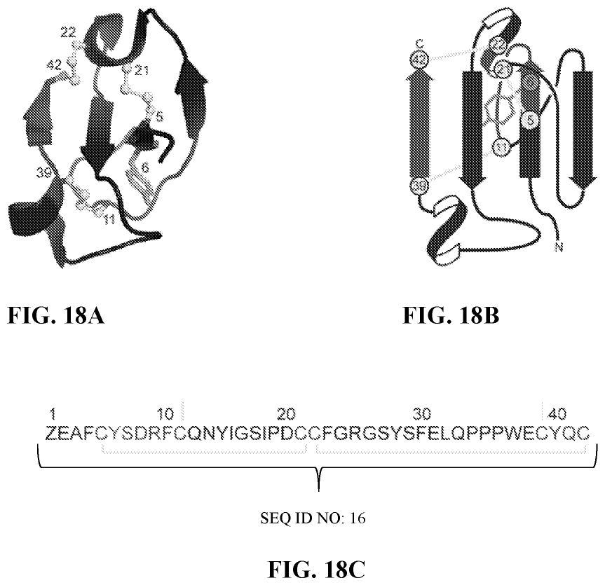

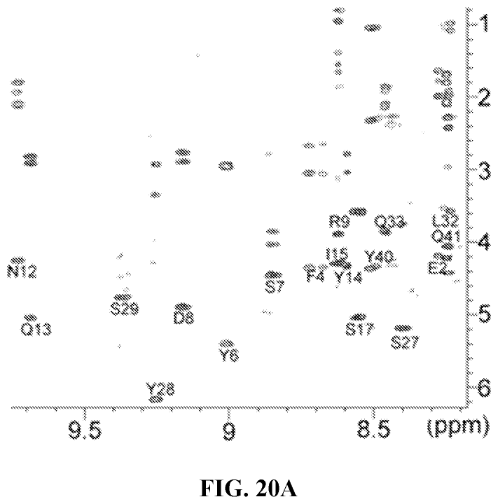

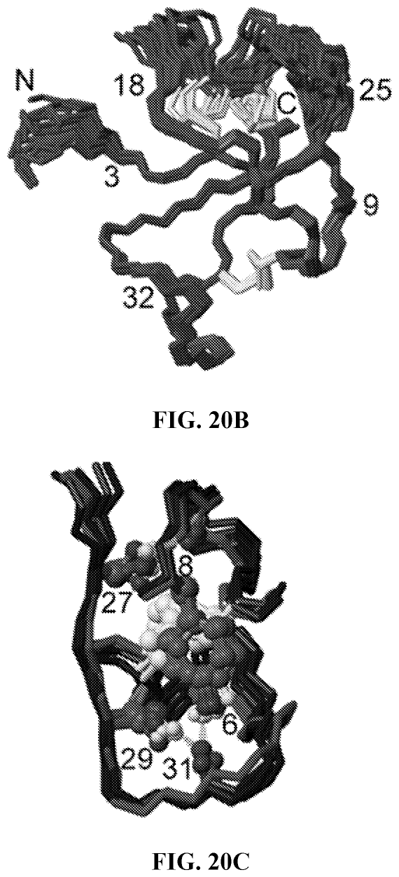

The recifin A secondary and tertiary structure is stabilized by three disulfide bonds, Cys5-Cys21, Cys22-Cys42 and Cys11-39, which provides a I-III, II-V, IV-VI arrangement of the cysteine bonds. FIG. 20A shows a superposition of TOCSY spectra of native and synthetic recifin A. FIG. 20B shows a solution NMR structure of [Phe⁶] recifin showing disulfides. Peptides with three difsulfide bonds often form topologically complex arrangements referred to as cystine knots, in which two disulfide bonds and their interconnecting backbone form a ring through which the third disulfide bond is threaded. However, what is unique about the recifin A structure is that all three disulfide bonds together with backbone segments form a ring that wraps around the third β-strand (residues 27-29). The fold of the peptide is stabilized by Tyr6, which is deeply buried in the peptide core and locked in place by interactions with surrounding residues (FIGS. 18A-C).

In summary, the 42-residue peptide recifin A was sequenced, the disulfide connectivity elucidated, and the unique three-dimensional structure of the peptide was solved using homonuclear solution state NMR spectroscopy. Recifin A was also synthetically made and found to be stable during many different laboratory conditions. The isolated peptide recifin A is shown to specifically modulate the enzymatic activity of full-length TDP1, but not an enzymatically active N-terminal truncated variant (Δ147TDP1) lacking the regulatory domain, suggesting an allosteric recifin A binding site within the regulatory domain of TDP1.

### Peptides

An embodiment of the invention provides a knotted cyclic peptide comprising, or consisting of, the amino acid sequence of SEQ ID NO: 11

(CX₁X₂XXXCXXXXXXXXXCCXXXXXXSXXLXXXXXXXXC), wherein X, X₁ and X₂ can be any amino acid provided that at least one of X₁ and X₂ is tyrosine, phenylalanine, or alanine. In an embodiment, X₁ is tyrosine, phenylalanine, or alanine. In an embodiment, X₂ is tyrosine, phenylalanine, or alanine. In an embodiment, X₁ is tyrosine. In an embodiment, X₁ is phenylalanine. In an embodiment X₁ is alanine.

In an embodiment, the peptides are isolated. The term “isolated,” as used herein, means having been removed from its natural environment.

In another embodiment, the peptides are purified. The term “purified,” as used herein, means having been increased in purity, wherein “purity” is a relative term, and not to be necessarily construed as absolute purity. For example, the purity can be about 50% or more, about 60% or more, about 70% or more, about 80% or more, about 90% or more, or about 100%. The purity preferably is about 90% or more (e.g., about 90% to about 95%) and more preferably about 98% or more (e.g., about 98% to about 99%).

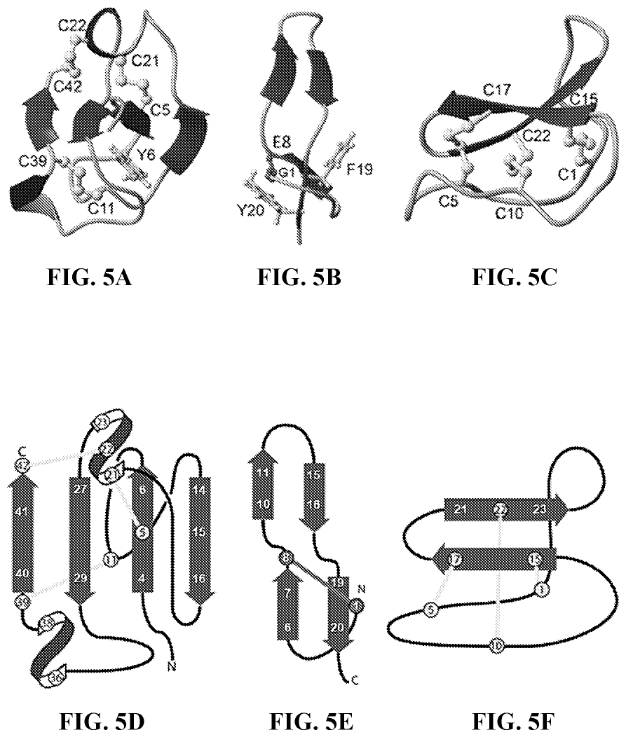

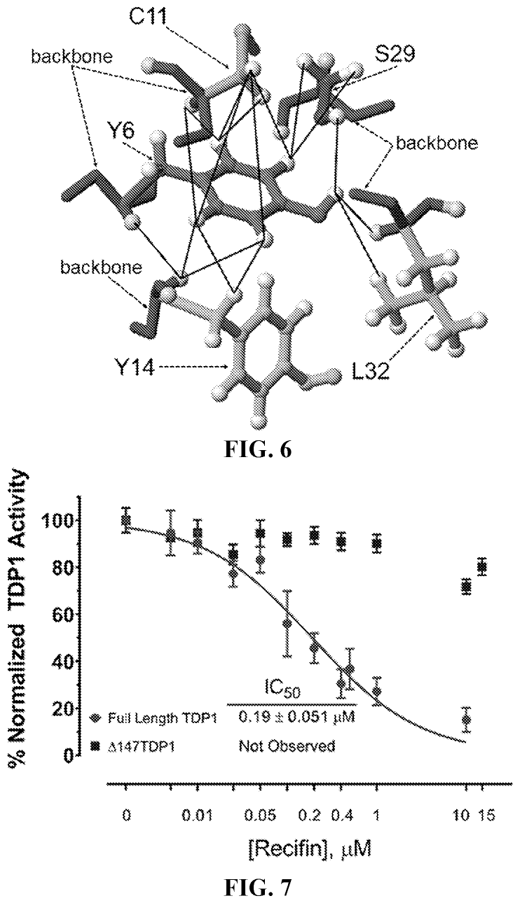

The peptides of the present invention may also comprise a four strand anti-parallel β-sheet and two helical turns. In an embodiment, the peptide may comprise a disulfide bond network that creates an embedded ring structure. In an embodiment, the peptide may comprise one, two, three, or four disulfide bonds. In an embodiment, the peptide may comprise three disulfide bonds, e.g., Cys I-III, Cys II-V, and Cys IV-VI, wherein Cys I refers to the first cysteine of SEQ ID NO: 11, Cys II refers to the second cysteine of SEQ ID NO: 11, Cys III refers to the third cysteine of SEQ ID NO: 11, Cys IV refers to the fourth cysteine of SEQ ID NO: 11, Cys V refers to the fifth cysteine of SEQ ID NO: 11, and Cys VI refers to the sixth cysteine of SEQ ID NO: 11. In an embodiment, the peptide may be in a configuration wherein the peptide may be stabilized by three disulfide bonds Cys I-III, Cys II-V, and Cys IV-VI, forming a ring together with two of the β-strands. In an embodiment, the peptide may be in a configuration wherein the ring that is formed by the three disulfide bonds (Cys I-III, Cys II-V, and Cys IV-VI) and the two β-strands is penetrated by a third β-strand. In an embodiment, the peptide may be in a configuration wherein the peptide may stabilized by a central tyrosine residue “locking” the structure in place (e.g. FIGS. 5A, 5D, and 6).

In an embodiment, the peptide comprises, consists essentially of, or consists of, SEQ ID NO: 7 (CYXXXXCXXXXXXXXXCCXXXXXXSXXLXXXXXXCXXC), wherein X can be any amino acid. This embodiment corresponds to a peptide comprising SEQ ID NO: 11, wherein Xi of SEQ ID NO: 11 is tyrosine, X₂ of SEQ ID NO: 11 is any amino acid, and the X residues are any amino acids.

In an embodiment, the peptide comprises, consists essentially of, or consists of, the amino acid sequence of SEQ ID NO: 8

(CYSXXXCXXYXGSXXXCCXXXXSYSXELXXXPWXCYXC), wherein X is any amino acid. Without being bound to any particular theory, the amino acids required in SEQ ID NO: 8 may be involved in the knotted cyclic shape of the peptides.

In an embodiment, the peptide comprises, consists essentially of, or consists of, the amino acid sequence of SEQ ID NO: 9

(CYXXRFCXXXXXXXXXCCXXRXXXSXXLXXXXWXCXXC), wherein X is any amino acid. Without being bound to any particular theory, the amino acids required in SEQ ID NO: 9 may be involved in the knotted cyclic shape of the peptides and/or interact with the regulatory domain of TDP1.

In an embodiment, the peptide comprises, or consists of, the amino acid sequence of SEQ ID NO: 12 (CYSXRFCXXYXGSXXXCCXXRXSYSXELXXXPWXCYXC), wherein X is any amino acid. Without being bound to any particular theory, the amino acids required in SEQ ID NO: 12 may be involved in the knotted cyclic shape of the peptides and/or interact with the regulatory domain of TDP1.

In an embodiment, the peptide comprises SEQ ID NO: 1, SEQ ID NO: 2, or SEQ ID NO: 16. In an embodiment, the peptide comprises SEQ ID NO: 1, SEQ ID NO: 2, or SEQ ID NO: 16, optionally with 1, 2, 3, 4, 5, or 6 amino acid substitutions, additions, or deletions. In an embodiment, the peptide is synthetically synthesized and comprises SEQ ID NO: 2 or SEQ ID NO: 16. In an embodiment, the peptide is synthetically synthesized and comprises SEQ ID NO: 1, SEQ ID NO: 2, or SEQ ID NO: 16, optionally with 1, 2, 3, 4, 5, or 6 amino acid substitutions, additions, or deletions. In an embodiment, the peptide comprises SEQ ID NO: 1, optionally with 1, 2, 3, 4, 5, or 6 amino acid substitutions. In an embodiment, the peptide comprises SEQ ID NO: 2, optionally with 1, 2, 3, 4, 5, or 6 amino acid substitutions. In an embodiment, the peptide comprises SEQ ID NO: 16, optionally with 1, 2, 3, 4, 5, or 6 amino acid substitutions. In an embodiment, the substitutions, additions, or deletions, as applicable in the above embodiments, are not at the position of the cysteine residues of the sequence.

In an embodiment, the peptide comprises the amino acid sequence of SEQ ID NO: 1, 2, or 16, optionally with 1, 2, 3, 4, 5, or 6 amino acid substitutions, and with an N-terminus truncation of 1, 2, 3, or 4 amino acids. In this regard, the peptide may comprise, consist essentially of, or consist of,

SEQ ID NO: 10
(EAFCYSDRFCQNYIGSIPDCCFGRGSYSFELQPPPWECYQC),
SEQ ID NO: 13
(AFCYSDRFCQNYIGSIPDCCFGRGSYSFELQPPPWECYQC),
SEQ ID NO: 14
(FCYSDRFCQNYIGSIPDCCFGRGSYSFELQPPPWECYQC),
or
SEQ ID NO: 15
(CYSDRFCQNYIGSIPDCCFGRGSYSFELQPPPWECYQC).

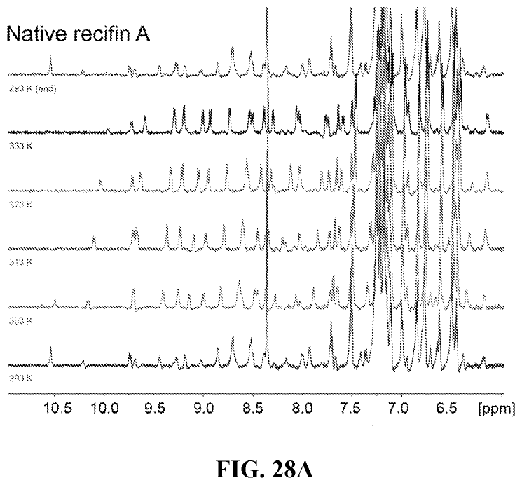

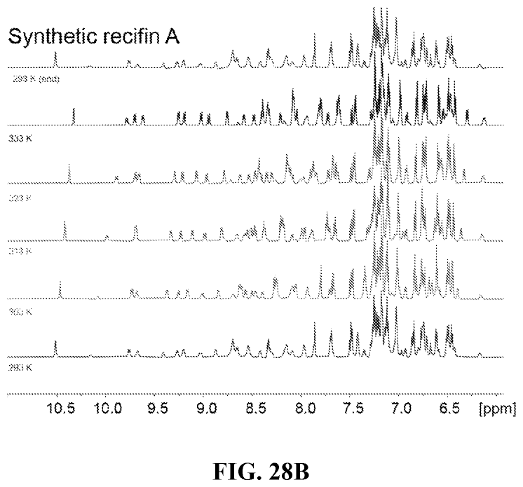

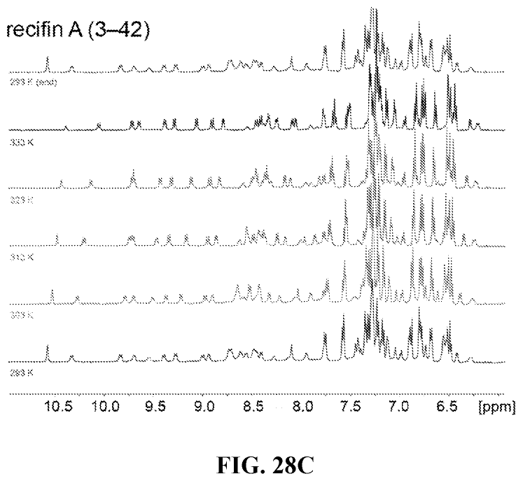

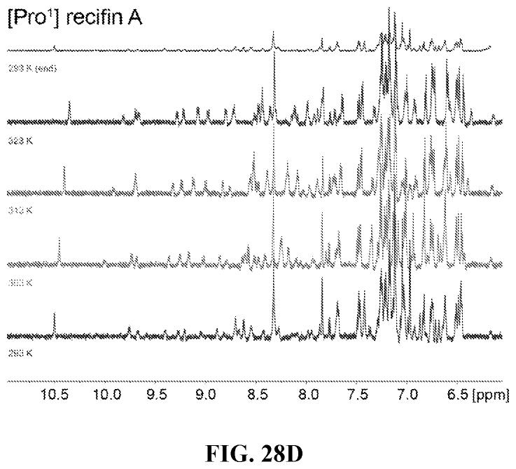

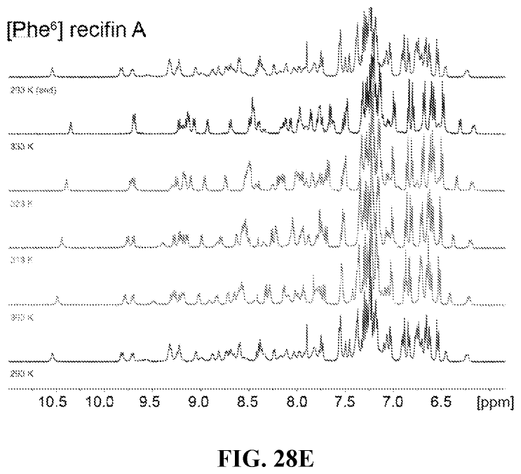

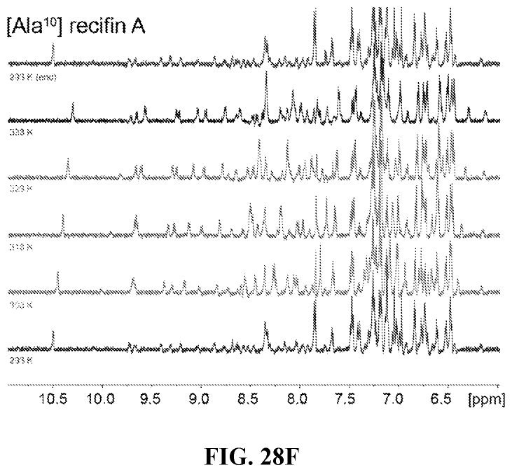

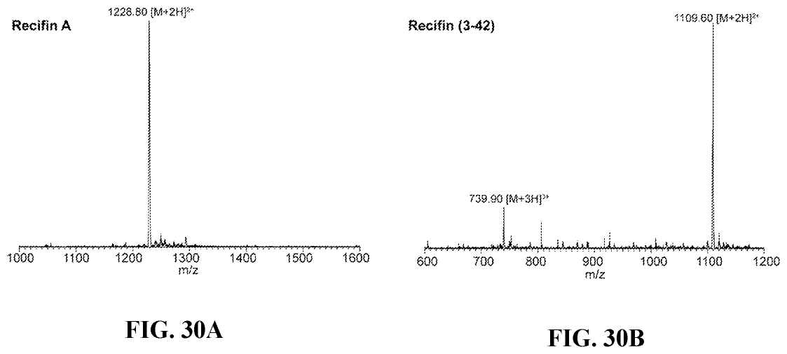

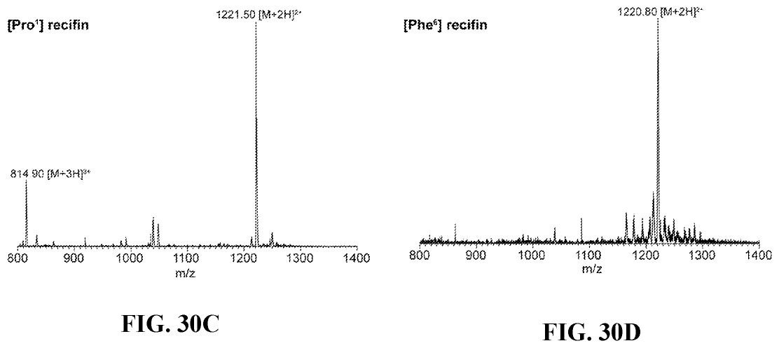

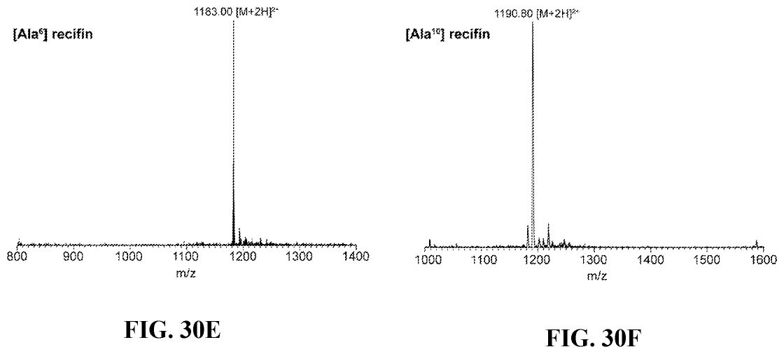

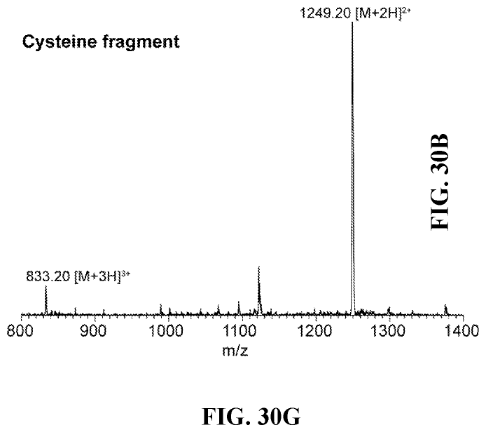

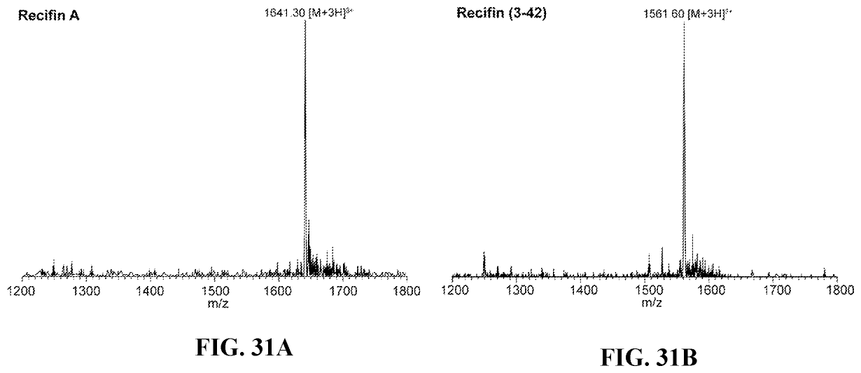

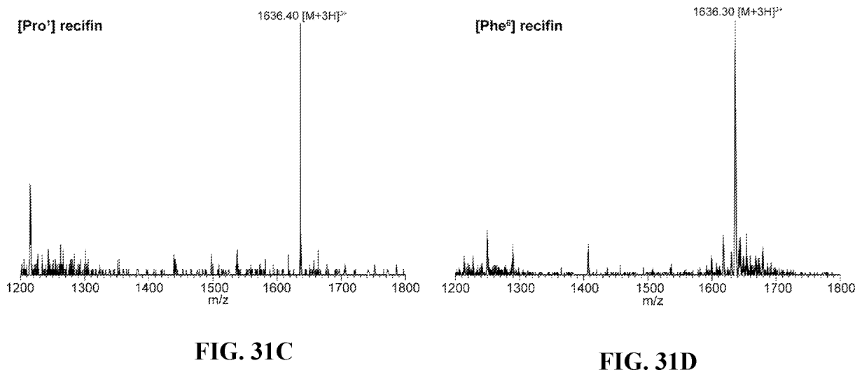

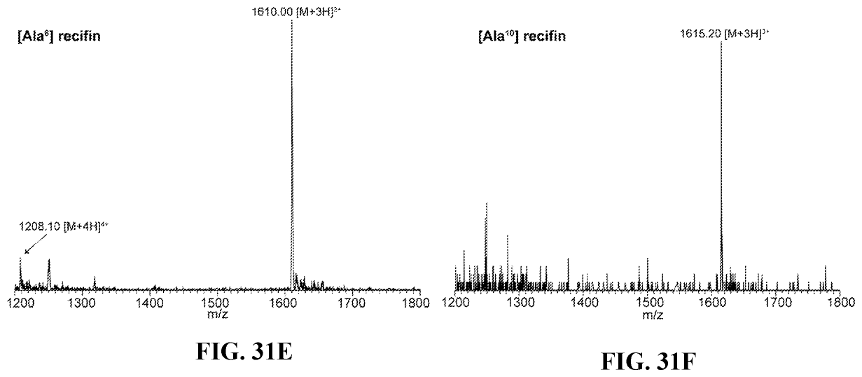

In some embodiments, the peptide comprises SEQ ID NO: 16 (ZEAFCYSDRFCQNYIGSIPDCCFGRGSYSFELQPPPWECYQC) with one or more of the following modifications:
(a) deletion of residue 1, residues 1 and 2, residues 1-3, or residues 1-4; or substitution Z 1P;
(b) Y6F or Y6A;
(c) R9A;
(d) F10A;
(e) E31R;
(f) P35A; and/or
(g) E38R;
   wherein Z is glutamine or glutamic acid (i.e., glx); and number refers to the positions of the amino acids residues in SEQ ID NO: 16. In some embodiments, the peptide comprises SEQ ID NO: 16 (ZEAFCYSDRFCQNYIGSIPDCCFGRGSYSFELQPPPWECYQC) with one or more of the following modifications:
(a) deletion of residue 1, residues 1 and 2, residues 1-3, or residues 1-4; or substitution Z1P;
(b) Y6F or Y6A; and/or
(c) F10A;
   wherein Z is glutamine or glutamic acid (i.e., glx); and number refers to the positions of the amino acids residues in SEQ ID NO: 16. FIG. 28A to 28F show thermal stability (298-333 K) of native recifin A, synthetic recifin A and synthetic recifin A analogues carried out using nuclear magnetic resonance on a 500 or 700 MHz Bruker Avance III equipped with a cryo probe. FIGS. 30A-30G show ES-MS spectra of recifin A and its analogues hydrazide fragments, as well as the cysteine fragment used for native chemical ligation. FIG. 31A-31F show ES-MS spectra of ligated recifin A and analogues. Table 7 shows FL-TDP1 inhibitory activity of recifin A and certain analogues.

In some embodiments, the peptide is not a naturally occurring peptide. Thus, for instance, in some embodiments the peptide can comprise a non-naturally occurring amino acid sequence, or is modified by the inclusion of additional moieties (e.g., PEG, cell penetrating peptides, or other modifications known in the art examples of which are described herein) to provide a peptide that is non-naturally occuring. In addition, or alternatively, in some embodiments the peptide does not comprise the entirety of the amino acid sequence of a naturally occurring peptide. For instance, in some embodiments, the peptide can comprise SEQ ID NO: 1, 2, or 16 with one or more (e.g., 1, 2, 3, 4, 5, or 6) substitutions, additions, or deletions. For instance, the peptide can comprise an amino acid sequence with about 85% to about 99% sequence identity (e.g, about 90-99% sequence identity or about 95-99% sequence identity) to SEQ ID NO: 1, 2, or 16 provided it includes at least one amino acid modification as compared to SEQ ID NO: 1. In some embodiments, the peptide comprises SEQ ID NO: 1, 2, or 16 with such modification (e.g., 1-6 substitutions, additions, or deletions), but still retains the amino acids specified in SEQ ID NO: 11, or in SEQ ID NO: 7, 8, 9, or 12 as described herein.

In an embodiment, the peptide comprises the amino acid sequence of recifin fragment SEQ ID NO: 4 (pyroglutamic acid EAFCYSDR).

In an embodiment, the peptide comprises the amino acid sequence of recifin fragment SEQ ID NO: 5 (FCQNYIGSIPDCCFGR).

In an embodiment, the peptide comprises the amino acid sequence of recifin fragment SEQ ID NO: 6 (GSYSFELQPPPQCQC).

An embodiment of the invention provides an isolated or purified peptide comprising, consisting essentially of, or consisting of, SEQ ID NO: 1, 2, or 16, or the amino acid sequence of SEQ ID NO: 1, 2, or 16 with 1, 2, 3, 4, 5, or 6 amino acid substitutions, additions, or deletions. In an embodiment, the peptide retains the amino acids specified in the amino acid sequence of SEQ ID NO: 7. In an embodiment, the peptide retains the amino acids specified in the amino acid sequence of SEQ ID NO: 8. In an embodiment, the peptide retains the amino acids specified in the amino acid sequence of SEQ ID NO: 9. In an embodiment, the peptide retains the amino acids specified in the amino acid sequence of SEQ ID NO: 12. The amino acids can be substituted, deleted, or inserted by any known suitable means, including by site mutagenesis. In some embodiments, the modifications to the amino acid sequence of SEQ ID NO: 1, 2, or 16 consist of amino acid substitutions.

In some embodiments, the peptide can comprise the amino acid sequence of SEQ ID NO: 1, 2, or 16 with 1, 2, 3, 4, 5, or 6 conservative amino acid substitutions. Conservative amino acid substitutions are known in the art and include amino acid substitutions in which one amino acid having certain chemical and/or physical properties is exchanged for another amino acid that has the same chemical or physical properties. For instance, the conservative amino acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc.

Alternatively or additionally, the peptides can comprise the amino acid sequence of SEQ ID NO: 1, 2, or 16 with 1, 2, 3, 4, 5, or 6 non-conservative amino acid substitutions. In this case, it is preferable for the non-conservative amino acid substitution to not interfere with or inhibit the biological activity and 3D structure of the peptides. Preferably, the non-conservative amino acid substitution enhances the biological activity of the peptides, such that the biological activity of the peptide is increased as compared to the parent peptide.

The peptides of the invention can comprise synthetic amino acids in place of one or more naturally-occurring amino acids. Such synthetic amino acids are known in the art and include, for example, aminocyclohexane carboxylic acid, norleucine, α-amino n-decanoic acid, homoserine, S-acetylaminomethyl-cysteine, trans-3-and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, omithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

The peptides of the invention can be further modified. For instance, the peptides can be glycosylated, amidated, carboxylated, phosphorylated, esterified, N-acylated, cyclized via, e.g., a disulfide bridge, or converted into an acid addition salt and/or optionally dimerized or polymerized, or conjugated.

In an embodiment, the peptide is modified by addition of a cell-penetrating peptide sequence. In a further embodiment, the cell-penetrating peptide sequence is at the N-terminus of the peptide. Cell-penetrating peptides assist with the delivery of peptides. Cell-penetrating peptides typically are composed of 5-30 amino acids and are usually positively charged at physiological pH due to the presence of several arginine and/or lysine residues. Any suitable cell-penetrating peptide may be used, for example, PENETRATIN, R8, TAT, TRANSPORTAN, and XENTRY.

In an embodiment, the peptide is modified by addition of at least one ethylene glycol ((CH₂OH)₂) group (e.g., polyethylene glycol). In a further embodiment, the at least one ethylene glycol is at the N-terminus of the peptide. In an embodiment, the at least one ethylene glycol is a polyethylene glycol of formula H—(O—CH2—CH2)ₙ—OH, wherein n can be from about 100 to about 800 (e.g., from about 150 to about 750, from about 200 to about 700, from about 250 to about 650, from about 300 to about 600, from about 350 to about 550, from about 400 to about 500, from about 420 to about 480, from about 440 to about 460, or about 450). In this regard, the at least one ethylene glycol is a polyethylene glycol and comprises from about 100 to about 800 ethylene glycols, from about 150 to about 750 ethylene glycols, from about 200 to about 700 ethylene glycols, from about 250 to about 650 ethylene glycols, from about 300 to about 600 ethylene glycols, from about 350 to about 550 ethylene glycols, from about 400 to about 500 ethylene glycols, from about 420 to about 480 ethylene glycols, from about 440 to about 460 ethylene glycols, or about 450 ethylene glycols.

In an embodiment, the at least one ethylene glycol is a polyethylene glycol and has a molecular weight from about 5 kDaltons to about 40 kDaltons. In this regard, the the at least one ethylene glycol has a molecular weight of from about 6 kDaltons to about 38 kDaltons, from about 8 kDaltons to about 35 kDaltons, from about 9 kDaltons to about 32 kDaltons, from about 11 kDaltons to about 30 kDaltons, from about 13 kDaltons to about 28 kDaltons, from about 15 kDaltons to about 25 kDaltons, from about 18 kDaltons to about 22 kDaltons, or about 20 kDaltons.

The polyethylene glycol can be linear or branched. A branched polyethylene glycol is defined herein as two or more polyethylene glycol chains linked to a common center. In contrast, a linear polyethylene glycol defined herein as a polyethylene glycol that does not have any chains linked to a common center.

### Pharmaceutical Compositions

An embodiment of the invention provides pharmaceutical compositions comprising (a) the peptide of the present invention described herein (referred to as “inventive molecule”) and (b) a pharmaceutically acceptable carrier. The inventive peptides, nucleic acids, recombinant expression vectors, host cells (including populations thereof), and populations of cells, all of which are collectively referred to as “inventive molecules” hereinafter, can be formulated into a composition, such as a pharmaceutical composition. In this regard, the invention provides a pharmaceutical composition comprising any of the inventive molecules, and a pharmaceutically acceptable carrier. The pharmaceutical composition containing any of the inventive molecules can comprise more than one inventive molecules, e.g., a peptide and a nucleic acid. Alternatively, the pharmaceutical composition can comprise inventive molecules in combination with one or more other pharmaceutically active agents or drugs, such as a chemotherapeutic agents, e.g., a topoisomerase I inhibitor, asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the pharmaceutical composition comprises a topoisomerase I inhibitor such as Camptothecin (CPT) or an analogue thereof (e.g., Topotecan, Irinotecan, Silatecan, Cositecan, Exatecan, Lurtotecan, Gimatecan, Belotecan, Rubitecan, CRLX101, or the like).

Preferably, the carrier is a pharmaceutically acceptable carrier. With respect to pharmaceutical compositions, the carrier can be any of those conventionally used and is limited only by chemico-physical considerations, such as solubility and lack of reactivity with the active compound(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

The choice of carrier will be determined in part by the particular inventive molecules, as well as by the particular method used to administer the inventive molecules. Accordingly, there are a variety of suitable formulations of the pharmaceutical composition of the invention. The following formulations for parenteral (e.g., subcutaneous, intravenous, intraarterial, intramuscular, intradermal, interperitoneal, and intrathecal) administration are exemplary and are in no way limiting. More than one route can be used to administer the inventive molecules, and in certain instances, a particular route can provide a more immediate and more effective response than another route.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The inventive molecules can be administered in a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-1,3-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quatemary ammonium salts, and (e) mixtures thereof.

The parenteral formulations will typically contain from about 0.5% to about 25% by weight of the inventive molecules material in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described. The requirements for effective pharmaceutical carriers for parenteral compositions are well-known to those of ordinary skill in the art (see, e.g., Lloyd et al. (eds.), Remington: The Science and Practice of Pharmacy, 22nd Ed., Pharmaceutical Press (2012)).

It will be appreciated by one of skill in the art that, in addition to the above-described pharmaceutical compositions, the inventive molecules of the invention can be formulated as inclusion complexes, such as cyclodextrin inclusion complexes, or liposomes.

For purposes of the invention, the amount or dose of the inventive molecules administered should be sufficient to effect a desired response, e.g., a therapeutic or prophylactic response, in the mammal over a reasonable time frame. For example, the dose of the inventive molecules should be sufficient to inhibit growth of a target cell or treat or prevent cancer in a period of from about 2 hours or longer, e.g., 12 to 24 or more hours, from the time of administration. In certain embodiments, the time period could be even longer. The dose will be determined by the efficacy of the particular inventive molecules and the condition of the mammal (e.g., human), as well as the body weight of the mammal (e.g., human) to be treated.

Many assays for determining an administered dose are known in the art. An administered dose may be determined in vitro (e.g., cell cultures) or in vivo (e.g., animal studies). For example, an administered dose may be determined by determining the IC₅₀ (the dose that achieves a half-maximal inhibition of symptoms), LD₅₀ (the dose lethal to 50% of the population), the ED₅₀ (the dose therapeutically effective in 50% of the population), and the therapeutic index in cell culture and/or animal studies. The therapeutic index is the ratio of LD₅₀ to ED₅₀ (i.e., LD₅₀/ED₅₀).

The dose of the inventive molecules also will be determined by the existence, nature, and extent of any adverse side effects that might accompany the administration of a particular inventive molecules. Typically, the attending physician will decide the dosage of the inventive molecules with which to treat each individual patient, taking into consideration a variety of factors, such as age, body weight, general health, diet, sex, inventive molecules to be administered, route of administration, and the severity of the condition being treated. By way of example and not intending to limit the invention, the dose of the inventive molecules can be about 0.001 to about 1000 mg/kg body weight of the subject being treated/day, from about 0.01 to about 10 mg/kg body weight/day, about 0.01 mg to about 1 mg/kg body weight/day, from about 1 to about to about 1000 mg/kg body weight/day, from about 5 to about 500 mg/kg body weight/day, from about 10 to about 250 mg/kg body weight/day, about 25 to about 150 mg/kg body weight/day, or about 10 mg/kg body weight/day.

The inventive molecules may be assayed for cytotoxicity by assays known in the art. Examples of cytotoxicity assays include a WST assay, which measures cell proliferation using the tetrazolium salt WST-1 (reagents and kits available from Roche Applied Sciences), as described in International Patent Application Publication WO 2011/032022.

In an embodiment, the concentration of the peptides of the invention in the pharmaceutical composition is at least 0.05 mg/ml (e.g., at least about 0.1 mg/ml, at least about 0.2 mg/ml, at least about 0.5 mg/ml, or at least about 1 mg/ml). This concentration is greater than the naturally occurring concentration of the peptides in their natural environment (e.g., in a sea sponge).

In an embodiment, the pharmaceutical composition comprises the peptide of the present invention that is modified with a cell-penetrating peptide sequence as described herein. In a further embodiment, the pharmaceutical composition comprises the peptide of the present that is modified with a cell-penetrating peptide sequence at the N-terminus.

In an embodiment, the pharmaceutical composition comprises the peptide of the present invention that is modified with at least one ethylene glycol (e.g., PEG) as described herein. In an embodiment, the pharmaceutical composition comprises the peptide of the present invention with at least one ethylene glycol (e.g., PEG) at the N-terminus. In still other embodiments, the pharmaceutical composition comprises the peptide described herein formulated with a delivery agent, such as a liposome or nanoparticle (e.g., lipid or polymer nanoparticle).

### Treatment Methods

An embodiment of the invention provides a peptide or pharmaceutical composition of the present invention for use in treating or preventing cancer. Without being bound by a particular theory or mechanism, it is believed that the peptides inhibit the cleavage of phosphodiester bonds by TDP1.

Another embodiment of the invention provides methods of treating or preventing cancer in a mammal, the method comprising administering to the mammal the peptide or pharmaceutical composition of the present invention in an amount effective to treat or prevent cancer in the mammal.

A further embodiment of the invention provides methods of inhibiting the cleavage of phosphodiester bonds by enzyme TDP1 in a mammal, the method comprising administering to the mammal the peptide or the pharmaceutical composition of the present invention in an amount effective to treat or prevent cancer in the mammal.

In an embodiment, the uses and methods herein further comprise administering to the mammal a topoisomerase I inhibitor, simultaneously or sequentially in any order with the peptide provided herein. Any topoisomerase I inhibitor can be used including, for instance, Camptothecin (CPT) and analogues thereof (e.g., Topotecan, Irinotecan, Silatecan, Cositecan, Exatecan, Lurtotecan, Gimatecan, Belotecan, Rubitecan, CRLX101, and the like).

In an embodiment, the peptide is at a concentration during use that inhibits the cleavage of phosphodiester bonds by enzyme TDP1 by at least 15% (e.g., by about 20%, about 25%, about 30%, about 35%, about 40%, about 45%, about 50%, about 55%, about 60%, about 65%, about 70%, about 75%, about 80%, about 85%, about 90%, about 95%, or about 100%).

The terms “treat” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, or a symptom or condition thereof.

With respect to the inventive methods, the cancer can be any cancer, including any of adrenal gland cancer, sarcomas (e.g., synovial sarcoma, osteogenic sarcoma, leiomyosarcoma uteri, angiosarcoma, fibrosarcoma, rhabdomyosarcoma, liposarcoma, myxoma, rhabdomyoma, fibroma, lipoma, and teratoma), lymphomas (e.g., small lymphocytic lymphoma, Hodgkin lymphoma, and non-Hodgkin lymphoma), hepatocellular carcinoma, glioma, head cancers (e.g., squamous cell carcinoma), neck cancers (e.g., squamous cell carcinoma), acute lymphocytic cancer, leukemias (e.g., hairy cell leukemia, myeloid leukemia (acute and chronic), lymphatic leukemia (acute and chronic), prolymphocytic leukemia (PLL), myelomonocytic leukemia (acute and chronic), and lymphocytic leukemia (acute and chronic)), bone cancer (osteogenic sarcoma, fibrosarcoma, malignant fibrous histiocytoma, chondrosarcoma, Ewing's sarcoma, malignant lymphoma (reticulum cell sarcoma), multiple myeloma, malignant giant cell tumor, chordoma, osteochondroma (osteocartilaginous exostoses), benign chondroma, chondroblastoma, chondromyxoid fibroma, osteoid osteoma, and giant cell tumors), brain cancer (astrocytoma, medulloblastoma, glioma, ependymoma, germinoma (pinealoma), glioblastoma multiforme, oligodendroglioma, schwannoma, and retinoblastoma), fallopian tube cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva (e.g., squamous cell carcinoma, intraepithelial carcinoma, adenocarcinoma, and fibrosarcoma), myeloproliferative disorders (e.g., chronic myeloid cancer), colon cancers (e.g., colon carcinoma), esophageal cancer (e.g., squamous cell carcinoma, adenocarcinoma, leiomyosarcoma, and lymphoma), cervical cancer (cervical carcinoma and pre-invasive cervical dysplasia), gastric cancer, gastrointestinal carcinoid tumor, hypopharynx cancer, larynx cancer, liver cancers (e.g., hepatocellular carcinoma, cholangiocarcinoma, hepatoblastoma, angiosarcoma, hepatocellular adenoma, and hemangioma), lung cancers (e.g., bronchogenic carcinoma (squamous cell, undifferentiated small cell, undifferentiated large cell, and adenocarcinoma), alveolar (bronchiolar) carcinoma, bronchial adenoma, chondromatous hamartoma, small cell lung cancer, non-small cell lung cancer, and lung adenocarcinoma), malignant mesothelioma, skin cancer (e.g., melanoma, basal cell carcinoma, squamous cell carcinoma, Kaposi's sarcoma, nevi, dysplastic nevi, lipoma, angioma, dermatofibroma, and keloids), multiple myeloma, nasopharynx cancer, ovarian cancer (e.g., ovarian carcinoma (serous cystadenocarcinoma, mucinous cystadenocarcinoma, endometrioid carcinoma, and clear cell adenocarcinoma), granulosa-theca cell tumors, Sertoli-Leydig cell tumors, dysgerminoma, and malignant teratoma), pancreatic cancer (e.g., ductal adenocarcinoma, insulinoma, glucagonoma, gastrinoma, carcinoid tumors, and VIPoma), peritoneum, omentum, mesentery cancer, pharynx cancer, prostate cancer (e.g., adenocarcinoma and sarcoma), rectal cancer, kidney cancer (e.g., adenocarcinoma, Wilms tumor (nephroblastoma), and renal cell carcinoma), small intestine cancer (adenocarcinoma, lymphoma, carcinoid tumors, Kaposi's sarcoma, leiomyoma, hemangioma, lipoma, neurofibroma, and fibroma), soft tissue cancer, stomach cancer (e.g., carcinoma, lymphoma, and leiomyosarcoma), testicular cancer (e.g., seminoma, teratoma, embryonal carcinoma, teratocarcinoma, choriocarcinoma, sarcoma, Leydig cell tumor, fibroma, fibroadenoma, adenomatoid tumors, and lipoma), cancer of the uterus (e.g., endometrial carcinoma), thyroid cancer, and urothelial cancers (e.g., squamous cell carcinoma, transitional cell carcinoma, adenocarcinoma, ureter cancer, and urinary bladder cancer). In a preferred embodiment, the cancer is a cancer that is characterized by the expression or overexpression of CD22 (such as, for example, hairy cell leukemia, CLL, PLL, non-Hodgkin's lymphoma, SLL, and ALL), BCMA (such as, for example, multiple myeloma and Hodgkin's lymphoma), or mesothelin (such as, for example, mesothelioma and ovarian and pancreatic adenocarcinoma).

As used herein, the term “mammal” refers to any mammal, including, but not limited to, mammals of the order Rodentia, including mice and hamsters, mammals of the order Logomorpha, including rabbits, mammals from the order Carnivora, including Felines (cats) and Canines (dogs), mammals from the order Artiodactyla, including Bovines (cows) and Swines (pigs), mammals from the order Perssodactyla, including Equines (horses), mammals of the order Primates, Ceboids, or Simoids (monkeys), and mammals of the order Anthropoids (humans and apes). An especially preferred mammal is the human.

### Nucleic Acids, Vectors, and Cells

An embodiment of the invention provides a nucleic acid encoding a peptide of the present invention. The term “nucleic acid,” as used herein, includes “polynucleotide,” “oligonucleotide,” and “nucleic acid molecule,” and generally means a polymer of DNA or RNA, which can be single-stranded or double-stranded, which can be synthesized or obtained (e.g., isolated and/or purified) from natural sources, which can contain natural, non-natural or altered nucleotides, and which can contain a natural, non-natural, or altered internucleotide linkage, such as a phosphoroamidate linkage or a phosphorothioate linkage, instead of the phosphodiester found between the nucleotides of an unmodified oligonucleotide. It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

Preferably, the nucleic acids of the invention are recombinant. As used herein, the term “recombinant” refers to (i) molecules that are constructed outside living cells by joining natural or synthetic nucleic acid segments, or (ii) molecules that result from the replication of those described in (i) above. For purposes herein, the replication can be in vitro replication or in vivo replication.

The nucleic acids can be constructed based on chemical synthesis and/or enzymatic ligation reactions using procedures known in the art. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, CO) and Synthegen (Houston, TX).

The nucleic acids of the invention can be incorporated into a recombinant expression vector. In this regard, the invention provides recombinant expression vectors comprising any of the nucleic acids of the invention. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally-occurring as a whole. However, parts of the vectors can be naturally-occurring. The inventive recombinant expression vectors can comprise any type of nucleotide, including, but not limited to DNA and RNA, which can be single-stranded or double-stranded, which can be synthesized or obtained in part from natural sources, and which can contain natural, non-natural or altered nucleotides. The recombinant expression vectors can comprise naturally-occurring, non-naturally-occurring internucleotide linkages, or both types of linkages. Preferably, the non-naturally occurring or altered nucleotides or internucleotide linkages does not hinder the transcription or replication of the vector.

The recombinant expression vector of the invention can be any suitable recombinant expression vector, and can be used to transform or transfect any suitable host cell. Suitable vectors include those designed for propagation and expansion or for expression or for both, such as plasmids and viruses. The vector can be selected from the group consisting of the pUC series (Fermentas Life Sciences), the pBluescript series (Stratagene, LaJolla, CA), the pET series (Novagen, Madison, WI), the pGEX series (Pharmacia Biotech, Uppsala, Sweden), and the pEX series (Clontech, Palo Alto, CA). Bacteriophage vectors, such as λGT10, λGT11, λZapII (Stratagene), λEMBL4, and λNM1149, also can be used. Examples of plant expression vectors include pBI01, pBI101.2, pBI101.3, pBI121 and pBIN19 (Clontech). Examples of animal expression vectors include pEUK-Cl, pMAM, and pMAMneo (Clontech). Preferably, the recombinant expression vector is a viral vector, e.g., a retroviral vector.

The recombinant expression vectors of the invention can be prepared using standard recombinant DNA techniques. Constructs of expression vectors, which are circular or linear, can be prepared to contain a replication system functional in a prokaryotic or eukaryotic host cell. Replication systems can be derived, e.g., from ColEl, 2 μ plasmid, λ, SV40, bovine papilloma virus, and the like.

Desirably, the recombinant expression vector comprises regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based.

The recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the inventive molecule (including functional portions and functional variants), or to the nucleotide sequence which is complementary to or which hybridizes to the nucleotide sequence encoding the molecule. The selection of promoters, e.g., strong, weak, inducible, tissue-specific, and developmental-specific, is within the ordinary skill of the artisan. Similarly, the combining of a nucleotide sequence with a promoter is also within the ordinary skill of the artisan. The promoter can be a non-viral promoter or a viral promoter, e.g., a cytomegalovirus (CMV) promoter, an SV40 promoter, an RSV promoter, or a promoter found in the long-terminal repeat of the murine stem cell virus.

The inventive recombinant expression vectors can be designed for either transient expression, for stable expression, or for both. Also, the recombinant expression vectors can be made for constitutive expression or for inducible expression.

Another embodiment of the invention further provides a host cell comprising any of the recombinant expression vectors described herein. As used herein, the term “host cell” refers to a cell that can contain the inventive recombinant expression vector. For purposes of producing a recombinant inventive molecule, the host cell is preferably a prokaryotic cell (e.g., a bacteria cell), e.g., an E. coli cell.

Also provided by the invention is a population of cells comprising at least one host cell described herein. The population of cells can be a heterogeneous population comprising the host cell comprising any of the recombinant expression vectors described, in addition to at least one other cell, e.g., a host cell which does not comprise any of the recombinant expression vectors. Alternatively, the population of cells can be a substantially homogeneous population, in which the population comprises mainly (e.g., consisting essentially of) host cells comprising the recombinant expression vector. The population also can be a clonal population of cells, in which all cells of the population are clones of a single host cell comprising a recombinant expression vector, such that all cells of the population comprise the recombinant expression vector. In one embodiment of the invention, the population of cells is a clonal population of host cells comprising a recombinant expression vector as described herein.

### Methods of Preparation

The peptides can be prepared by any of a number of conventional techniques. The peptides can be isolated or purified from a recombinant source. For instance, a DNA fragment encoding a desired a peptide can be subcloned into an appropriate vector using well-known molecular genetic techniques. The fragment can be transcribed and the polypeptide subsequently translated in vitro. Commercially available kits also can be employed. The polymerase chain reaction optionally can be employed in the manipulation of nucleic acids. An embodiment of the invention provides methods of preparing the peptides of the present invention by expressing a nucleic acid encoding the peptide in a host cell. In an embodiment, the nucleic acid is in a vector. In an embodiment, the host cell is notE. coli.

The peptides also can be synthesized using an automated peptide synthesizer in accordance with methods known in the art. Alternately, the peptides can be synthesized using standard peptide synthesizing techniques well-known to those of skill in the art (e.g., as summarized in Bodanszky, Principles of Peptide Synthesis, (Springer-Verlag, Heidelberg: 1984)). In particular, the peptides can be synthesized using the procedure of solid-phase synthesis (see, e.g., Merrifield, J. Am. Chem. Soc., 85: 2149-54 (1963); Barany et al., Int. J. Peptide Protein Res., 30: 705-739 (1987); and U.S. Pat. No. 5,424,398, incorporated herein by reference). If desired, this can be done using an automated peptide synthesizer. Removal of the t-butyloxycarbonyl (t-BOC) or 9-fluorenylmethyloxycarbonyl (Fmoc) amino acid blocking groups and separation of the polypeptide from the resin can be accomplished by, for example, acid treatment at reduced temperature. The protein-containing mixture then can be extracted, for instance, with diethyl ether, to remove non-peptidic organic compounds, and the synthesized polypeptide can be extracted from the resin powder (e.g., with about 25% w/v acetic acid). Following the synthesis of the polypeptide, further purification (e.g., using HPLC) optionally can be performed in order to eliminate any incomplete proteins, polypeptides, peptides or free amino acids. Amino acid and/or HPLC analysis can be performed on the synthesized polypeptide to validate its identity.

In one embodiment, a peptide as described herein is provided by a method that comprises (a) synthesizing an N-terminal fragment of the peptide and synthesizing a C-terminal fragment of the peptide, (b) ligating the N-terminal fragment of the peptide to the C-terminal fragment of the peptide to provide the whole peptide, and (c) oxidizing the ligated peptide to induce folding.

The N-terminal and C-terminal fragments can be prepared by any method of peptide synthesis, such as the methods described above or other methods known in the art. Furthermore, the N-terminal and C-terminal fragments can be of any suitable length, provided the ligated fragments provide the entire length of the desired end product peptide. The N-terminal and C-terminal fragments can each be, for instance, 5-40 amino acids long, provided the ligated fragments provide the desired product.

Ligation of the N-terminal and C-terminal fragments can be performed by any suitable method (e.g., Zheng et al., Nature Protocols, 8: 2483-2495(2013)). In some embodiments, a hydrazide group can be provided on the N-terminal fragment, such as by incubating with NH₂NH₂. Ligation can then be performed by converting the hydrazide to an azide and reacting with the C-terminal peptide fragment.

The resulting peptide can be folded by inducing the formation of cysteine bonds between the cysteine residues of the peptide. Any suitable method can be used, for instance, by oxidation of the peptide through exposure to an oxidation buffer (e.g., ammonium bicarbonate buffer with reduced and oxidized glutathione).

The following examples further illustrate the invention but, of course, should not be construed as in any way limiting its scope.

### EXAMPLES

All purification solvents were of High Performance Liquid Chromatography (HPLC) and spectrophotometry grade. Mass spectrometry solvents were Liquid chromatography- mass spectrometry (LC-MS) grade and purchased from either Thermo Fisher Scientific (Waltham, MA) or Burdick & Jackson (Muskegon, MI). Mass spectrometry measurements were performed using an Accurate-Mass Quadrupole-Tof (Q-TOF) Dual-ESI 6530B instrument with an online 1260 Infinity binary HPLC system (Agilent Technologies, Inc., Santa Clara, CA), calibrated daily and operated with continual, internal calibration using reference mass ions at 121 and 1221 m/z. For MS, chromatographic separations were performed using linear gradients from 0-60% acetonitrile (0.1% v/v formic acid modified) at 1.00 mL/min on a POROSHELL 300SB-C18, 5 μm, 2.1×75 mm column (Agilent Technologies, Inc., Santa Clara, CA) maintained at 40° C. Source parameters for dual-electrospray ionization+(ESI+) were: capillary 4000 V, fragmentor 150-175 V, skimmer 65 V. Nitrogen flow was 12 L/min at 350° C. High-resolution measurements (minimum of resolution at 1521 m/z) were acquired in the range from 100-3200 m/z at a scan rate of 1 spectra/sec., and for MS/MS was 50-3200 m/z at a scan rate of 3 spectra/sec for both MS and MS/MS. Collision induced dissociation was accomplished using nitrogen gas and ramped collision energies (CE) calculated using the equation:

$$C E = \frac{4 \times ( \frac{m}{Z} )}{100}$$

### Example 1

This example demonstrates the extraction and isolation of recifin A.

The sponge Axinella sp., (Voucher #ID 0CDN7410, NSC #C020686) was harvested at a depth of 40 m at the Thunderbolt reef, south-southwest of Cape Recife Nature Reserve, Port Elizabeth, South Africa. A voucher specimen for this collection is maintained at the Smithsonian Institution (Suitland, MD). Aqueous extracts of Axinella sp. were provided by the Natural Products Branch of the National Cancer Institute and were prepared as previously reported (McCloud, Molecules, 15(7): 4526-63 (2010)). The dried extract was reconstituted in water at a concentration of 10 mg/mL and then subjected to vacuum-assisted chromatography using Bakerbond C₄ wide-pore media (Mallinckrodt Baker, Inc., Phillipsburg, NJ). Compounds were eluted using a stepwise methanol gradient of five column volumes (CV) each of 100% water, 40% methanol, 60% methanol and 100% methanol, and the resulting fractions were evaporated under vacuum and then lyophilized to dryness. A high-throughput biochemical assay for inhibition of TDP1 enzymatic activity was utilized to track fraction activity (Bermingham, et al., SLAS Discov., 2472555217717200 (2017)). Active fractions were subjected to RP-HPLC at room temperature, first using a DYNAMAX 300 Å, 5 μm, C4 column (Rainin, Woburn, MA), eluted with a 0-60% methanol gradient over 20 CV, and then purified to homogeneity using a VYDAC Protein&Peptide, 300 Å, 5 μm, C₁₈ column (Grace Davison Discovery Science, Deerfield, IL), eluted either with a 0-60% methanol, 20 CV gradient, or a 5-40%, 20 CV acetonitrile gradient. Purified peptides were lyophilized and stored at −20° C.

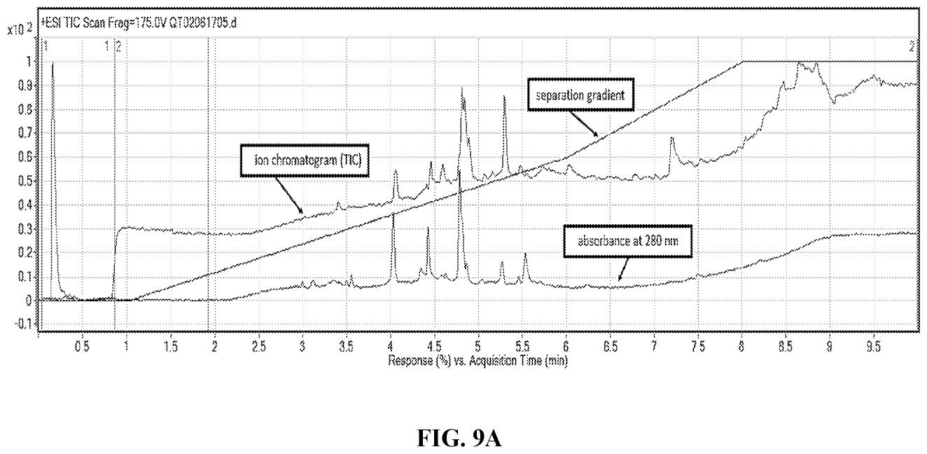

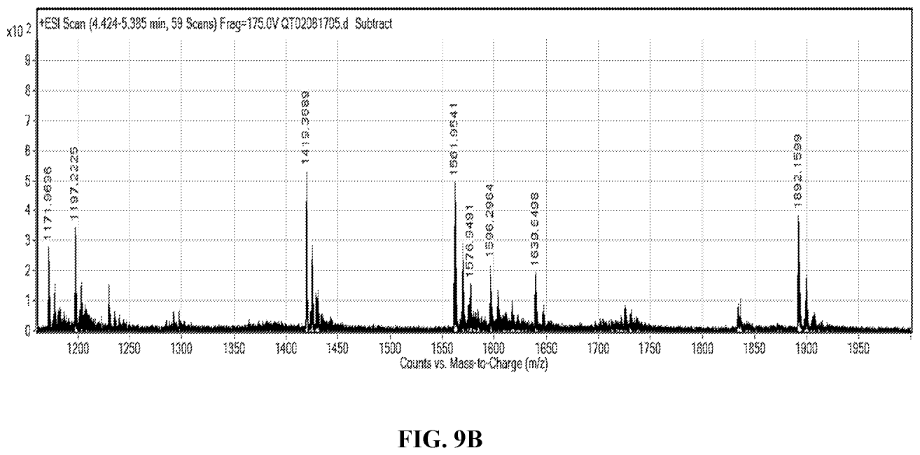

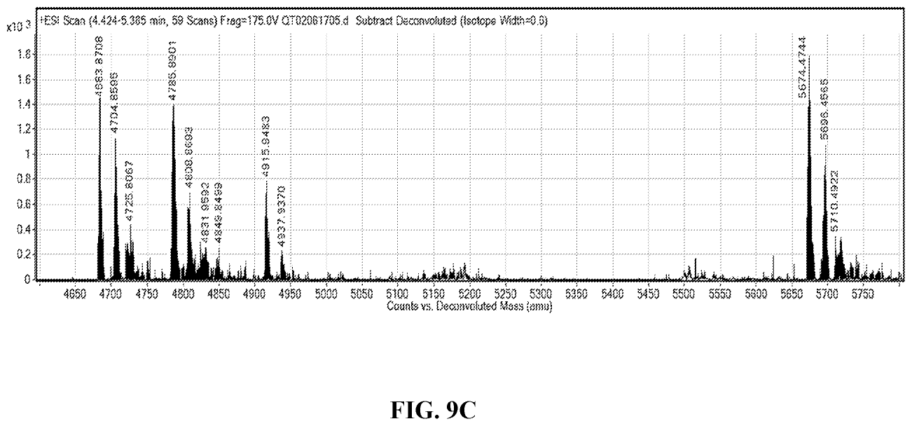

A family of four main Axinella peptides was isolated from the initial chromatographic step with observed average masses of 4683.87, 4785.89, 4915.95, and 5674.47 Da (FIGS. 9A-9C).

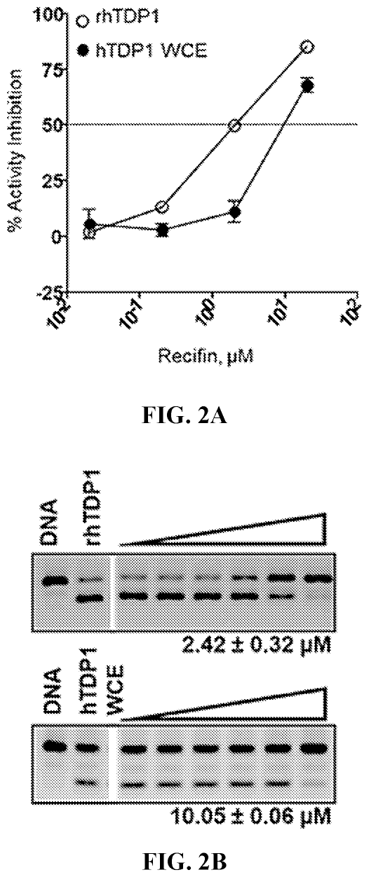

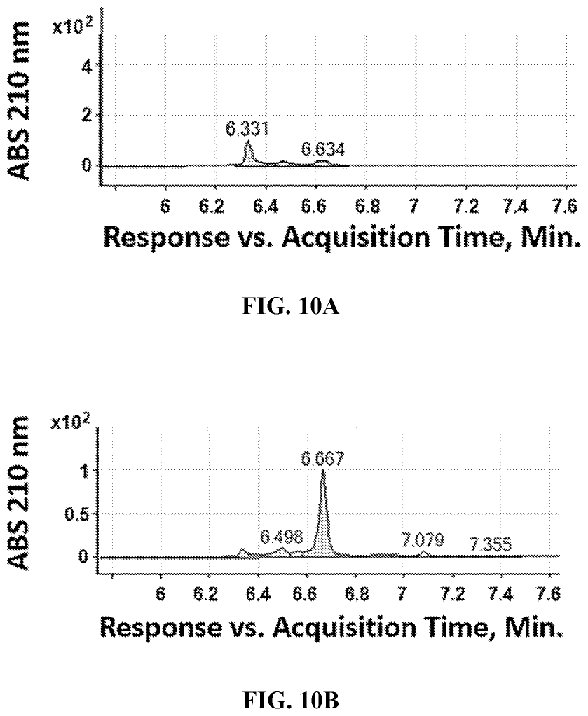

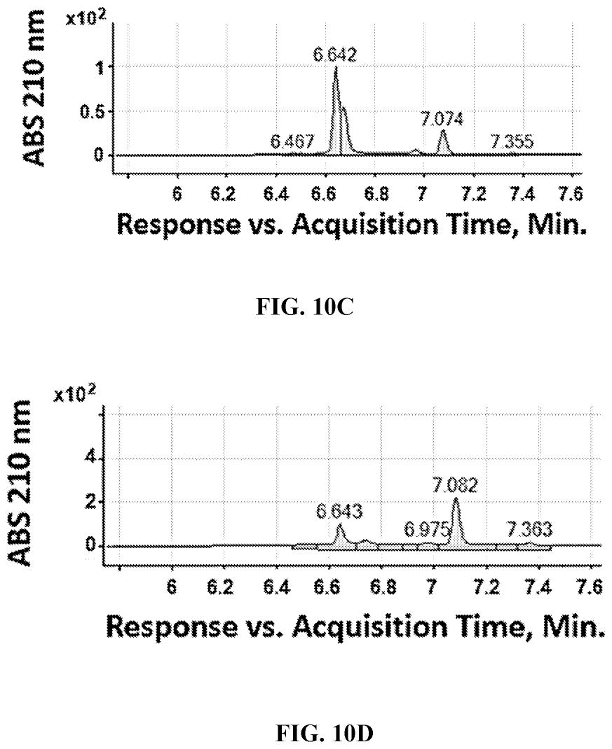

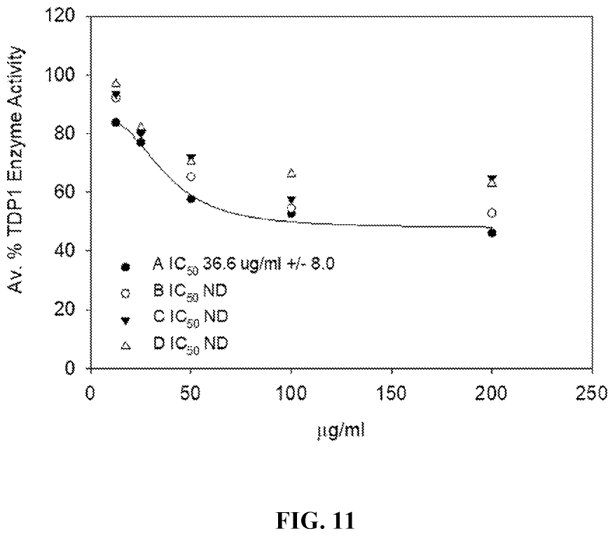

A combination of reversed-phase-high pressure liquid chromatography (RP-HPLC) and bioassay-guided fractionation was used to isolate the most abundant and most active peptide, recifin A (MW 4915.95 Da), to homogeneity. The yield of purified recifin A from the crude aqueous extract was approximately 0.1% w/w. Recifin A was found to inhibit full-length recombinant human TDP1 enzymatic activity in a concentration-dependent manner with an IC₅₀ of 2.4 μM in a biochemical assay for cleavage of a 5′-radiolabeled oligonucleotide DNA substrate containing a 3′-phosphotyrosyl residue (FIGS. 2A-2B). Recifin A retained the ability to inhibit TDP1 processing of the radiolabeled oligonucleotide within a whole-cell extract assay context, indicating the specificity and stability of the molecule. This is significant as it shows that recifin A could exert its inhibitory activity against TDP1 in the presence of other cellular macromolecules and against an enzyme whose regulatory domain had potentially been post-translationally modified. The other main Axinella-derived peptides showed weaker TDP1 inhibitory activity, indicating they are likely additional members of the same structural class of peptides (FIGS. 10A-D and FIG. 11; and Tables 1-4).

TABLE 1
(data from FIG. 10A)

| Peak | RT | Area | Area Sum % | Av. Mass, Da |
|---|---|---|---|---|
| 1 | 6.331 | 1010.78 | 50.26 | 4916 (Recifin A) |
| 3 | 6.472 | 350.22 | 17.41 | 4787 |
| 5 | 6.634 | 452.26 | 22.49 | 4787, 4916, 6463, 6899 |

TABLE 2
(data from FIG. 10B)

| Peak | RT | Area | Area Sum % | Av. Mass, Da |
|---|---|---|---|---|
| 1 | 6.337 | 883.62 | 15.32 | 4916 |
| 2 | 6.498 | 561.4 | 9.73 | 4786 |
| 3 | 6.564 | 256.53 | 4.45 | 4684 |
| 4 | 6.667 | 3273.91 | 56.77 | 4786 |
| 5 | 6.915 | 250.8 | 4.35 | 4684 |
| 7 | 7.079 | 258.96 | 4.49 | 5674 |

TABLE 3
(data from FIG. 10C)

| Peak | RT | Area | Area Sum % | Av. Mass, Da |
|---|---|---|---|---|
| 3 | 6.642 | 2375.43 | 44.36 | 4684 |
| 4 | 6.672 | 1372.86 | 25.64 | 4786 |
| 6 | 6.964 | 258.99 | 4.84 | 4684 |
| 7 | 7.074 | 797.37 | 14.89 | 5674 |

TABLE 4
(data from FIG. 10D)

| Peak | RT | Area | Area Sum % | Av. Mass, Da |
|---|---|---|---|---|
| 1 | 6.497 | 373.07 | 6.05 | 4684 |
| 2 | 6.643 | 1135.53 | 18.42 | 4684 |
| 3 | 6.738 | 509.02 | 8.26 | 4786 |
| 4 | 6.802 | 412.81 | 6.7 | 4786 |
| 5 | 6.912 | 267.69 | 4.34 | 4684 |
| 6 | 6.975 | 386.16 | 6.26 | 4684 |
| 7 | 7.082 | 2198.17 | 35.65 | 5674 |
| 9 | 7.363 | 555.98 | 9.02 | 5674 |

### Example 2

This example demonstrates the amino acid sequencing and disulfide assignments of recifin A.

Purified recifin A was dissolved in 0.25 M Tris HCl, 1 mM ethylenediaminetetraacetic acid (EDTA), 6 M guanidine HCl, reduced at room temperature with 2-mercaptoethanol and alkylated with 4-vinylpyridine according to standard techniques (Crimmins, et al., Curr. Protoc. Protein Sci., Chapter 11, Unit 11 (2005). The peptide was purified by RP-HPLC using a VYDAC C18 column and eluted using an acetonitrile gradient as described above. Reduced and alkylated peptide was subjected to digestion with various proteases per manufacturer's protocols (Roche Diagnostics, Indianapolis, IN): trypsin, chymotrypsin; (Thermo Scientific, Rockford, IL): glu-c; (Sigma-Aldrich, St. Louis, MO): proline specific endopeptidase; (Clontech Takara Bio USA, Inc., Mountain View, CA): pfu-pyroglutamate aminopeptidase). Peptide fragments were sequenced by MS/MS CID or purified by RP-HPLC and sequenced by automated N-terminal Edman degradation on an Applied Biosystems 494 protein sequencer (Applied Biosystems, Foster City, CA) according to manufacturer's protocols. PEAKS software version 7.5 was used for de novo peptide sequencing (Bioinformatics Solutions, Inc., Waterloo, ON, Canada). Precursor mass error tolerances were set to 5 ppm and fragment ion error tolerance was set to 0.1 Da.

Disulfide bonds were mapped using a partial reduction and sequential alkylation technique (Gray, Protein Sci., 2(10): 1732-48 (1993)). A quantity of 1 nmol recifin A (81 μM final concentration) was incubated in 0.1 M glycine HCl pH 2.5 with 5, 10, 20, or 50 mM TCEP at 37° C. for 30 min. N-ethylmaleimide, freshly prepared in acetonitrile, was added to the reaction to a final concentration of 250 mM and incubated at 37° C. for 15 min. Partially alkylated species were desalted and separated by RP-HPLC using a VYDAC Protein & Peptide, 300 Å, 5 μm, C18 column at 40° C., using a linear gradient of water with 0.05% (v/v) trifluoroacetic acid (TFA) to 50% acetonitrile with 0.05% (v/v) TFA. The partially reduced/alkylated species were either combined with 0.1 M tris-HCl pH 8.0, 1 M urea, and digested with chymotrypsin for 18 hr at room temperature, or fully reduced with 5 mM (dithiothreitol) DTT, alkylated with 14 mM iodoacetamide and digested with trypsin for 18 hours at 37° C. Fragments were sequenced by LC-MS/MS collision induced dissociation and PEAKS de novo sequencing software as described above. Intact disulfide-bridged peptides were analyzed by LC-MS and assigned using MassHunter qualitative analysis software with BioConfirm, version B.07.00 (Agilent Technologies, Inc., Santa Clara, CA). Input amino acid sequences of the disulfide isoforms were constructed with a fixed N-terminal pyroglutamic acid residue and amino acid numbers 22 and 42 were fixed as N-ethylmaleimide alkylated cysteine residues.

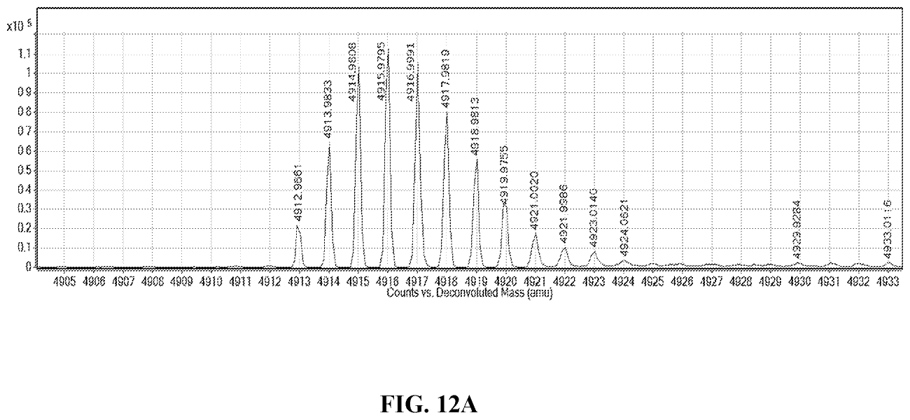

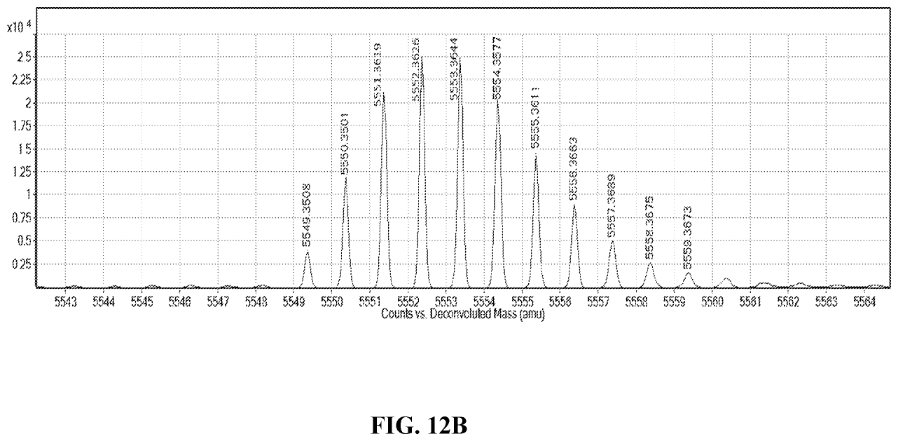

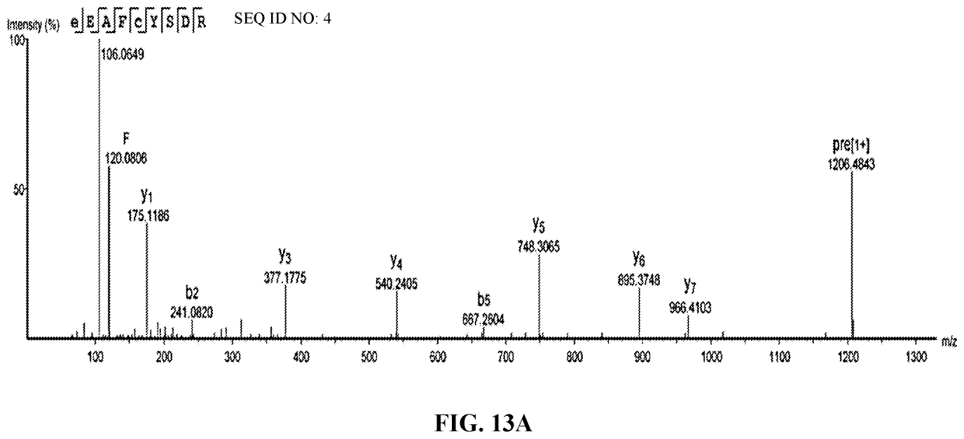

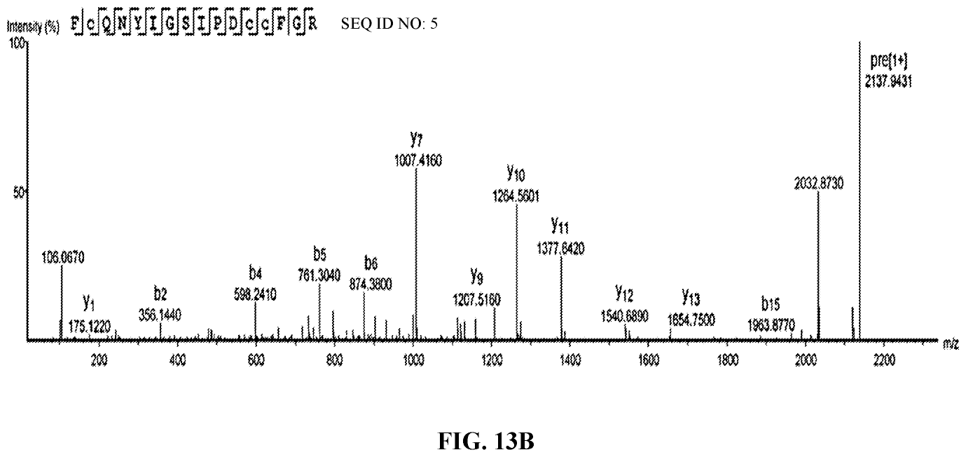

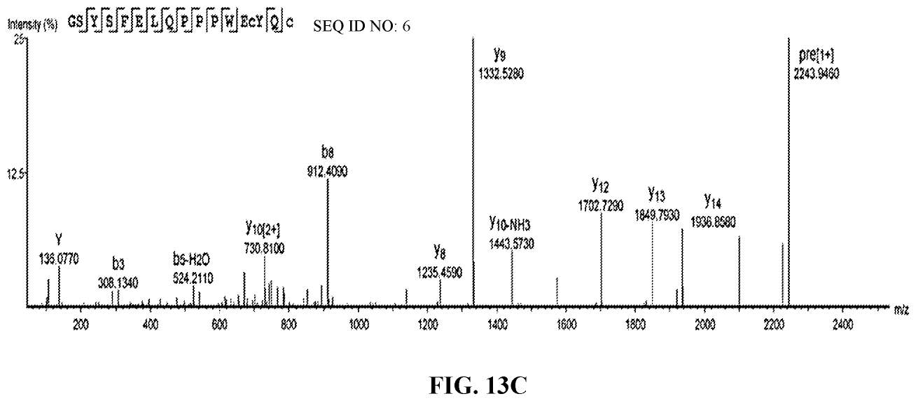

The monoisotopic mass of the intact recifin A peptide was observed at 636.38 Da, which indicated the conversion of six cysteine residues to S-pyridylethyl cysteine and three disulfide bonds (FIGS. 12A-12B). Neither the native peptide nor the 4-VP alkylated peptide was amenable to N-terminal amino acid sequencing by Edman degradation, which suggested a blocked N-terminus. A trypsin digest of the 4-VP alkylated peptide was performed which generated three fragments, A, B, and C with molecular weights of 1205.48, 2136.94, and 2242.95 Da, respectively (FIGS. 13A-13C).

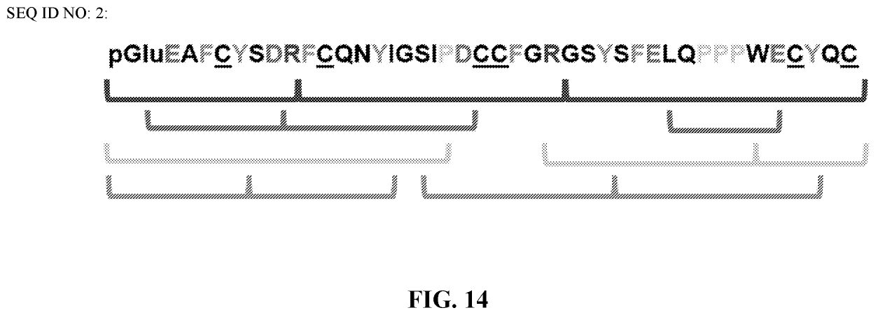

Sequencing of the tryptic fragments by LC/MS/MS and confirmation by Edman degradation (FIGS. 13A-13C) indicated the presence of a pyroglutamic acid residue (pGlu) on the N-terminus of Fragment A explaining the lack of success with N-terminal Edman degradation of recifin A. This was confirmed by selective cleavage of the pGlu with Pfu pyroglutamate aminopeptidase. Upon successful enzymatic removal of the N-terminal pGlu from the reduced, alkylated recifin A, 35 contiguous amino acids of the N-terminally-truncated peptide were able to be sequenced by Edman degradation. In addition to trypsin digestion, the alkylated peptide was subjected to digestion with chymotrypsin, glutamic acid C-terminal (Glu-C), and proline endopeptidases. The resultant fragments were sequenced by CID MS/MS only (FIG. 14) and confirmed the full sequence of recifin A. The theoretical mass of the proposed amino acid sequence of recifin A was 4918.9994 Da, which differed from the observed mass by 6.0333 Da, confirming the presence of three disulfide bonds (2.1 ppm mass error).

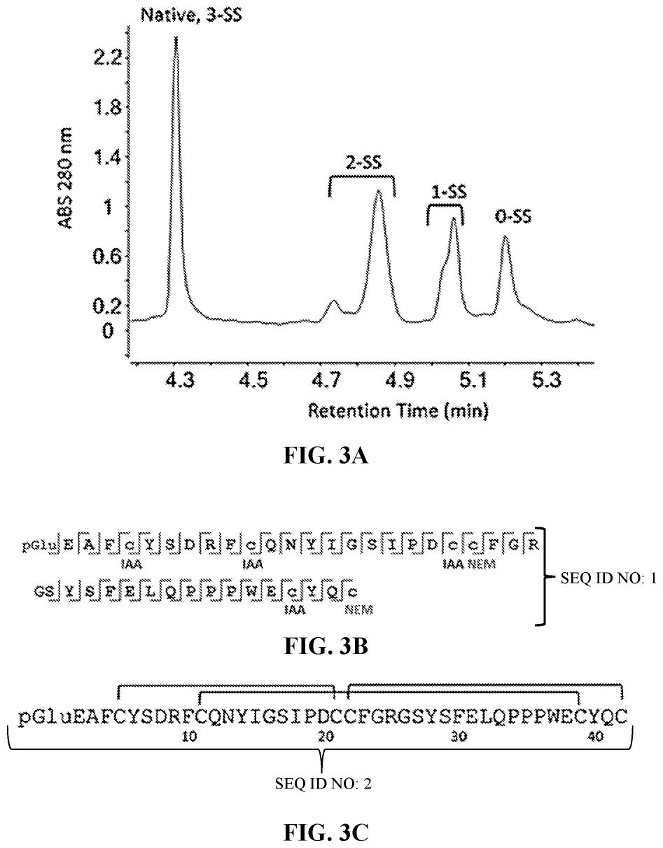

A combination of 80 μM recifin A and 50 mM Tris(2-carboxyethyl)phosphine (TCEP) yielded one (two intact cystines, 2-SS), and two disulfide bond (one intact cystine, 1-SS) reduction events and the fully-reduced species (zero intact cystines, 0-SS) as shown in FIG. 3A.

The main 2-SS, N-ethylmaleimide (NEM) alkylated peptide isoform was fully reduced, alkylated, and digested with trypsin. The resultant trypsin fragments were sequenced to map the positions of the alkylation events (FIGS. 3A and 3B).

NEM-alkylated cysteine residues were found to be at positions 22 and 42, which mapped a projected cystine linkage at Cys IV-VI. The 2-SS, NEM-alkylated peptide was digested with chymotrypsin to map the remaining, intact disulfide linkages by LC-MS. MassHunter (Agilent Technologies, Inc.) software was used to construct a database of the three possible disulfide-linked sequence permutations (Cys I-II, Cys Cys IV-VI; Cys I-III, Cys II-V, Cys IV-VI; and Cys I-V, Cys II-III, Cys IV-VI) and to match the observed chymotrypsin fragment masses to a set of theoretical digest fragment masses. A limitation of 5 ppm mass error was applied to the fragment matching process. Only fragments which linked Cys I-III and Cys II-V were observed (Table 6). Taken together, the data indicated the disulfide bond connectivity of recifin A to be Cys I-III, Cys II-V, and Cys IV-VI (FIG. 3C).

TABLE 5
Recifin A 2-SS NEM isoform observed chymotrypsin fragments

| Observed | | Theoretical | Mass Error, | | Cystine |
|---|---|---|---|---|---|
| Mass | m/z | Mass | ppm | Sequence | Linkage |
| 476.19 | 477.20 (z = 1) | 476.19 | 0.27 | pGlu-EAF | |
| 1360.51 | 681.26 (z = 2) | 1360.51 | −0.78 | CY + IGSIPDC [symbol] F | CysI-III |
| 1818.69 | 910.35 (z = 2) | 1818.70 | −1.24 | pGlu-EAFCY + IGSIPDC [symbol] F | CysI-III |
| 1880.75 | 627.93 (z = 3) | 1880.75 | −1.43 | CY + IGSIPDC [symbol] FGRGSY | CysI-III |
| 2289.94 | 764.32 (z = 3) | 2289.95 | −1.47 | SDRFCQNY + ELQPPPWECY | CysII-V |
| 2338.93 | 1170.47 (z = 2) | 2338.93 | −3.10 | pGlu-EAFCY + IGSIPDC [symbol] FGRGSY | CysI-III |
| 2524.04 | 842.35 (z = 3) | 2524.05 | −4.50 | SDRFCQNY + SFELQPPPWECY | CysII-V |
| 2646.05 | 883.03 (z = 3) | 2646.06 | −4.34 | SDRFCQNY + ELQPPPWECYQ [symbol] | CysII-V |
| 2880.15 | 961.06 (z = 3) | 2880.16 | −3.61 | SDRFCQNY + SFELQPPPWECYQ [symbol] | CysII-V |

In Table 5, the observed chymotrypsin digested recifin A 2-SS isoform peptides were matched to theoretical digest fragments of the three possible disulfide-linked amino acid sequence permutations. Recifin A amino acid sequence fixed modifications included N-terminal pyroglutamic acid (pGlu) and N-ethylmaleimide alkylated cysteines (C) Cys IV and VI. Mass error tolerance for matching was set to 5 ppm.

The molecular weight, number of cysteine residues, along with the stability of recifin A, is similar to that reported for members of the inhibitory cystine knot (ICK) family, comprising protease inhibitors, toxins, and anti-microbial peptides. However, the ICK family is characterized by the intertwined, or “knotted,” Cys I-IV, Cys II-V, Cys III-VI disulfide bond arrangement (Pallaghy, et al., Protein Sci., 3(10): 1833-9 (1994). The recifin A disulfide bond framework is Cys I-III, Cys II-V, and Cys IV-VI, so while recifin A is a CRP, the peptide is not a member of the ICK family. The primary amino acid sequence of recifin A is not homologous to any sequence within the non-redundant GenBank translated protein database (BLASTp search). Further, recifin A has no identified amino acid sequence alignments with Asteropus-derived CRPs (or ICK peptides) within the KNOTTIN database (Postic, et al., Nucleic Acids Res., 46(D1): D454-D458 (2018)).

### Example 3

This example demonstrates the NMR Spectroscopy and Structure Determination of recifin A.

All spectra were acquired on a 600 MHz Bruker AVANCE III equipped with a cryogenically cooled probe (Bruker Biospin, Billerica, MA). An approximately 2 mg sample of recifin A was dissolved in 90% H₂O/10% D₂O at pH 4.85 and 1D ¹H and 2D ¹H-¹H Total Correlated Spectroscopy (TOCSY) (mixing time 80 ms) and ¹H-¹H Nuclear Overhauser Effect Spectroscopy (NOESY) (mixing time 200 ms) experiments were acquired at 298K. In addition, a series of ¹H-¹H TOCSY experiments were acquired over 24 h, directly after adding lyophilized recifin A to 100% D₂O to investigate slow exchange of HN protons. This was followed by acquisition of ¹H-¹³C HSQC and ¹H-¹H NOESY (200 ms mixing time) experiments in 100% D₂O. TOPSPIN 3.5 (Bruker) was used to process the spectra, and the data were referenced to water at δ_{H} 4.76 ppm. Sequential assignments were completed using CCPNMR analysis 2.4.1 (CCPN, University of Cambridge, Cambridge, UK) and XEASY (Bartels, et al., J. Biomol. NMR., 6(1): 1-10 (1995)). Distance restraints were derived from ¹H-¹H NOESY experiments acquired in 90% H₂O/10% D₂O and 100% D₂O, and ϕ and ψ dihedral angle restraints were derived from chemical shifts from ¹H-¹H NOESY and ¹H-¹³C HSQC experiments analyzed by the online version of TALOS-N (Shen, et al., J Biomol NMR, 56(3): 227-41 (2013)) to derive ϕ and ψ dihedral angle restraints. χ1 and χ2 dihedral restraints for Cys residues were derived from DISH (Armstrong, et al., Chem. Sci., 9(31): 6548-6556 (2018)) and additional χ1 dihedral restraints were derived from a combination of TALOS-N, patterns of NOE intensities and preliminary structure calculations. Hydrogen bonds were introduced based on D₂O exchange data, or in the case of hydroxyl groups based on exchange behavior in the H₂O sample, and preliminary structure calculations. An initial 20 structures were calculated using the using automated assignments in CYANA (Guntert, Methods Mol. Biol., 278: 353-78 (2004); Guntert, et al., J Mol. Biol., 273(1): 283-98 (1997)). After manual assessment of the output all remaining NOEs could be unambiguously assigned. Structural refinement was carried out in a watershell using CNS (Linge, et al., Proteins, 50(3): 496-506 (2003)) where 50 structures were calculated and 20 representative structures selected based on MolProbity scores (Chen, et al., Acta Crystallogr. D. Biol. Crystallogr., 66(Pt 1): 12-21 (2010)) and energies. Root mean square deviations (RMSDs) were calculated using MOLMOL (Koradi, et al., J. Mol. Graph., 14(1): 51-5, 29-32 (1996)) and structural visualization was carried out using MOLMOL and PyMOL (the PyMOL Molecular Graphics System, Version 1.7.4, Schrödinger, LLC). Recifin A structure has been deposited into the PDB (Berman, et al., Nucleic Acids Res., 28(1): 235-42 (2000); ID 6XN9)), and NMR data have been deposited into the Biological Magnetic Resonance Bank Ulrich, et al., Nucleic Acids Res., 36 (Database issue), D402-8 (2008)) (ID 30767).

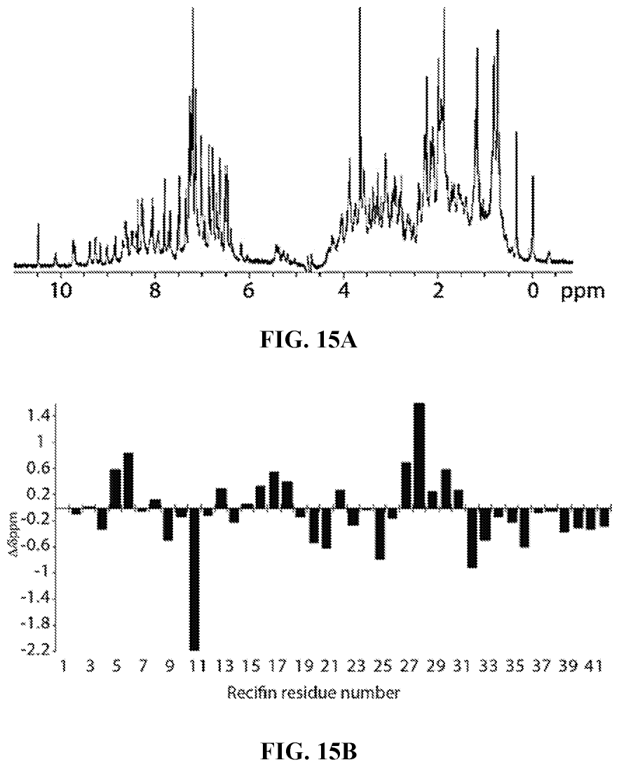

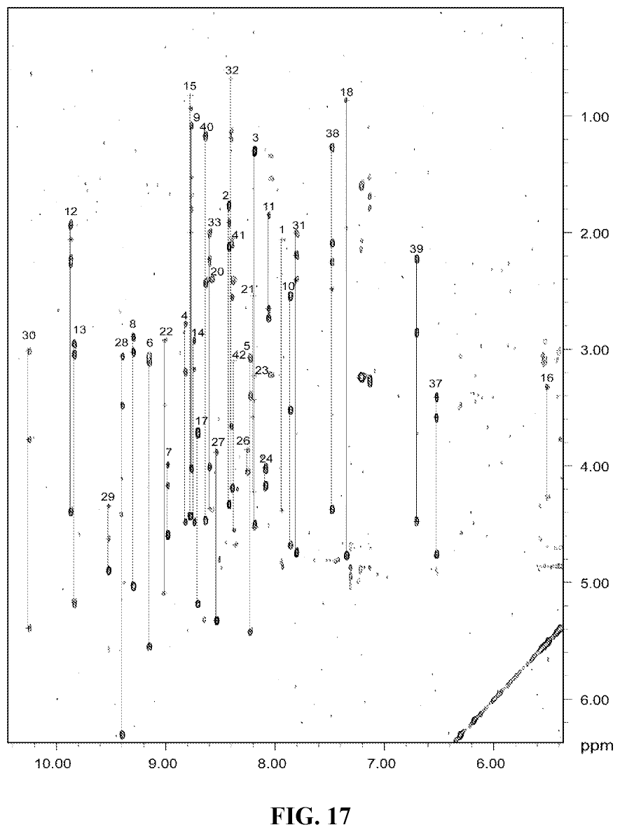

Given the lack of sequence homology to known proteins and unexpected disulfide array when compared to other CRPs, recifin A was subjected to solution NMR spectroscopy in an attempt to characterize its three-dimensional structure. The one-dimensional ¹H NMR spectrum showed excellent signal dispersion across the entire spectral region indicating a well-structured peptide (FIG. 15A). Homonuclear ¹H TOCSY (FIG. 17) and NOESY data were used for sequential assignments as described previously (Schroeder, et al., Methods Mol. Biol., 2068: 129-162 (2020)). This process proved a significant challenge due to a number of unusual chemical shifts and features in the NMR data for recifin A. Chemical shift anomalies included the Gly16 HN proton at 5.51 ppm, upfield of several Hα βprotons. The Hβ resonances of Tyr40 and Pro35 were observed at 1.15 and −0.33 ppm, respectively, the latter being the most upfield resonance in the spectrum. Finally, the Hα of Cys11 was essentially overlapping one of the Hβ resonance at 2.68 ppm. Resonances observed at 4.94 and 5.58 were, after identification of TOCSY peaks to their respective Hβ protons, assigned as the hydroxyl protons of Ser27 and Ser29, and a resonance at 7.96 as the phenolic proton of Tyr6 because of a lack of TOCSY peaks but strong NOESY connections to Tyr6 Hε. These protons are all not expected to be visible in the spectra due to fast exchange with the solvent, but in the recifin A structure must clearly be involved in strong hydrogen bonds and protected from the solvent. Finally, four individual aromatic ¹H signals were identified for Tyr6 (Hδ1, Hδ2, Hε1, Hε2) revealing that it is positioned in a tightly packed environment where ring-flips are sufficiently slowed down to prevent averaging into the typically observed single Hδ* and Hε* resonances. Line broadening, suggesting dynamics, was also observed around residues 21-25, with the HN proton of Arg25 broadened beyond detection. In addition to the homonuclear data, a ¹H-¹³C HSQC data set was recorded at natural abundance, which was essential for confirming all proton assignments and provided ¹³C chemical shift information for dihedral restraints.

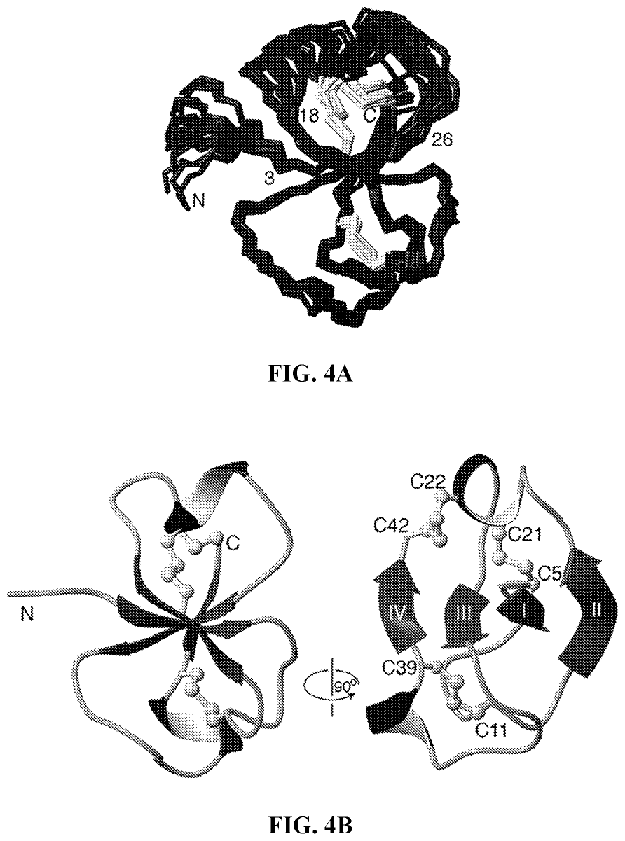

Initial analysis of secondary Hα chemical shifts suggested secondary structural features in form of short β-strands and α-helices/turns, as indicated by significant positive and negative shifts, respectively (FIG. 15B). The three-dimensional solution structure of recifin A was calculated using torsion angle dynamics in CYANA followed by refinement in a watershell using Crystallography and NMR System (CNS). A total of 425 distance restraints, including 403 distance restraints derived from NOEs, 22 hydrogen bond restraints, and 75 dihedral angle restraints (ϕ, ψ, χ) were included in the calculations (Table 6). A family of 20 structures were chosen to represent the solution structure of recifin A based on energies, stereochemical quality and consistency with the experimental data (Table 6). As seen from the superposition of the ensemble, the structure is well defined, except a loop region comprising residues, 21-25 consistent with the observed line broadening (FIGS. 4A and 4B). The structure is dominated by a central, antiparallel β-sheet comprising four strands involving residues 4-6,14-16,27-29 and 40-41, and two short 3₁₀ helical turns involving residues 21-23 and 36-38. The elements of secondary structure are stabilized by the three disulfide bonds, with the Cys5-Cys21 and Cys22-Cys42 disulfides bracing the 21-23 turn to strands 1 and 4, respectively, and the Cys11-Cys39 cross-bracing two loops. Intriguingly, the disulfides form an embedded ring together with their backbone segments, through which the third strand (27-29) is threaded. This arrangement gives rise to a previously not observed fold and represents a new type of cysteine-rich peptide knot. Although this is somewhat reminiscent of the inhibitory cystine knot, where two of the disulfide bonds form a ring structure through, which the third disulfide bond is threaded forming the knot (Daly, et al., Curr. Opin. Chem. Biol., 15(3): 362-8 (2011); Craik, Curr. Opin. Chem. Biol., 38: 8-16 (2017)), it bears perhaps even more resemblance to the lasso peptides, in which the peptide backbone is threaded through a ring formed by an N-terminus to side chain carboxyl lactam bond (FIGS. 5A-5F) (Maksimov, et al., Nat. Prod. Rep., 29(9): 996-1006 (2012)).

TABLE 6
Statistical analysis of the 20 best structural
models of recifin A based on MolProbity scores.

| Distance restraints | |
|---|---|
| Intraresidue (\|i − j\| = 0) | 126 |
| Sequential (\|i − j\| = 1) | 115 |
| Medium range (\|i − j\| ≤ 5) | 50 |
| Long range (\|i − j\| > 5) | 112 |
| Hydrogen bonds | 22 |
| Total | 425 |
| Dihedral angle restraints | |
| Φ | 25 |
| φ | 25 |
| χ | 25 |
| Total | 75 |
| Structure statistics | |
| Energies (kcal/mol, mean ± SD) | |
| Overall | −1422.3 ± 36.5 |
| Bonds | 16.9 ± 1.2 |
| Angles | 49.8 ± 4.4 |
| Improper | 19.3 ± 2.4 |
| Dihedral | 181.4 ± 1.6 |
| Van de Waals | −221.2 ± 3.9 |
| Electrostatic | −1469.1 ± 35.2 |
| NOE (experimental) | 0.03 ± 0.01 |
| Constrained dihedrals (experimental) | 0.6 ± 0.3 |
| Atomic RMSD (Å) | |
| Mean global backbone (1-42)^{a} | 0.93 ± 0.31 |
| Mean global heavy (1-42)^{a} | 1.57 ± 0.26 |
| Mean global backbone (3-17, 27-42) | 0.44 ± 0.08 |
| Mean global heavy (3-17, 27-42) | 1.17 ± 0.15 |
| MolProbity statistics | |
| Clash score, all atoms^{b} | 10.02 ± 1.9 |
| Poor rotamers | 0 ± 0 |
| Favoured rotamers | 95.8 ± 1.6 |
| Ramachandran outliers (%) | 0 ± 0 |
| Ramachandran favoured (%) | 94.9 ± 3.3 |
| MolProbity^{c} score | 1.8 ± 0.2 |
| MolProbity percentile | 82.9 ± 8.3 |
| Violations | |
| Distance constraints (>0.5 Å) | 0 |
| Dihedral-angle constraints (>5°) | 0 |

^{a}Pairwise RMSD from 20 refined structures over amino acids 1-42
^{b}Number of steric overlaps (>0.4 Å)/1000 atoms
^{c}100% is the best among structures of comparable resolution. 0% is the worst.

However, the embedded ring in recifin A (FIG. 5A) is bigger than both lasso-peptides (e.g., microcin J25) and prototypic ICK peptides (e.g., kalata B1) (FIG. 5B and 5C, respectively; see also FIGS. 5E and 5F, respectively). The unusual fold of recifin A is further stabilized by Tyr6, which is deeply buried in the middle of the peptide (FIG. 6), and locked in place by a number of residues, most notably Cys11, Tyr14, Ser29, and Leu32. It is because of this tight packing that Tyr6 does not undergo the usual fast “ring flips” typically observed for aromatic residues, where only one resonance line and set of NOEs can be observed for each of the geminal Hδ* and Hε* protons. Instead, recifin A has extensive NOES from surrounding residues to both Hδ1/2 and Hε1/2 protons locking Tyr6 in a specific conformation. In addition, a series of NOEs from the phenolic proton of Tyr6 to other surrounding residues can be observed, further highlighting the structurally stabilizing role of Tyr6 as these types of NOEs are rarely seen in a NOESY spectrum. The buried Tyr6 phenol group serves both as hydrogen bond donor, to the backbone carbonyl of Glu31, and as hydrogen bond acceptor for the HN proton of Gln33, while the hydroxyl groups of Ser27 and Ser29 serve as hydrogen bond donors to the carbonyls of Asp8 and Glu31, respectively. Ring current effects from aromatic residues are responsible for the unusual chemical shifts with Tyr6 packing against the Hα of Cys11, while the positioning of the side chains of Tyr14, Tyr28 and Trp37 are consistent with ring current effects on the HN of Gly16, and the F113 resonances of Tyr40 and Pro35, respectively. This is an unprecedented structural arrangement.

One side of recifin A has a patch of residues known to be involved in protein-protein interactions, including Arg9, Phe10, Arg25 and Trp37, and this region may be the binding interface with regulatory domain of TDP1.

### Example 4

This example demonstrates the biological activity and kinetics of recifin A.

TDP1 enzymatic activity inhibition assays using a radiolabeled oligonucleotide DNA substrate were carried out as previously described (Marchand, et al., Mol. Cancer Ther., 13(8): 2116-26 (2014)). Briefly, serial dilutions of recifin A were incubated with 1 nM 5′-32P-labeled DNA oligos (P14Y: 5′-[32P]-GATCTAAAAGACTT(3′-pTyr)-3′) (SEQ ID NO: 3), 30 pM recombinant human TDP1 or 2 μg/mL of hTDP1 WCE which were collected from TDP1 knockout (TDP1−/−) DT40 cells complemented with human TDP1. The reactions were carried out in a final volume of 10 μL in 1×LMP 1 reaction buffer (50 mM Tris-HCl, pH 7.5, 80 mM KCl, 2 mM EDTA, 1 mM DTT, 40 μg/mL BSA, 0.01% TWEEN 20) at room temperature for 15 minutes and terminated by adding 10 μ−L of 2×stop buffer (99.5% formamide, 10 mM EDTA, 0.01% methylene blue, 0.01% bromophenol blue). A 20% DNA sequencing gel was used to load the samples and exposed to a PHOSPHORIMAGER screen for further analysis by TYPHOON FLA 9500 (GE Healthcare).

FRET-based TDP1 enzymatic activity inhibition assays were carried out as previously described (Bermingham, et al., SLAS Discov., 2472555217717200 (2017)). Briefly, for Michaelis-Menten analysis, an eight-point FRET substrate concentration response was used (from 0.01-3 μM substrate) in the presence of 0, 0.2, 0.5, 1, and 2 μM recifin A. Quadruplicate reactions were setup in which a 1.25× concentration of either full-length TDP1 or Δ1-147TDP1 was diluted to 1× by the addition of a 6× solution of substrate and recifin A to reach a final concentration of 0.5 nM TDP1 (full length or truncated) and the indicated substrate and recifin A concentration in 1× Phosphate Buffered Saline (PBS) pH 7.4, 80 mM potassium chloride, 1 mM TCEP, referred to as “1× TDP1 buffer.” After dilution these reactions were transferred to a black small volume 384-well plate (Greiner Bio-One, Monroe, NC). Fluorescence measurements (excitation: 520 nM, emission: 550 nm) were taken at 30 sec intervals for 1 h using a i3x SpectraMax plate reader (Molecular Devices, Sunnyvale, CA). Reaction progression curves for each condition were examined for linearity over the time course and the reaction rate for each condition was determined by linear regression using GraphPad Prism software (version 8.3.1, San Diego, CA). Reaction rates were replotted in terms of substrate concentration, and kinetic parameters for each recifin A treatment concentration were calculated by non-linear regression (GraphPad Prism) according to the following equation:

$$v = \frac{{V}_{max} [ S ]}{{K}_{m} + [ S ]}$$

For IC₅₀ determinations, a 12-point concentration response curve was prepared over a recifin A concentration range of 0-15 μM. This was accomplished by diluting a 5X stock solution of recifin A and TDP1 FRET substrate into a stock solution of 1.25× TDP1 buffer containing 0.625 nM full-length TDP1 or Δ147TDP1, bringing the final concentration to 1× TDP1 buffer, 0.5 nM enzyme (or a no enzyme control), 1 μM FRET substrate, and 0-15 μM recifin A. Reactions were setup in triplicate using the same plates and plate reader described above for the kinetic measurements. Reaction wells were read at 0 (T₀) and 15 (T₁₅) min after initiation. The T₁₅ data was background corrected by subtracting T₀ fluorescence measurements. Corrected data was normalized to a control with no enzyme present (0% activity) and a vehicle control (100% activity). Recifin A concentrations were converted to logio-values and normalized data were fitted to the following equation by nonlinear regression (least squares fit with a variable slope) and an IC₅₀ value was calculated using GraphPad Prism software:

$$\% Normalized Activity = \frac{100}{( 1 + 1 {0}^{( ( log {IC}_{50} - [ R e c i f i n ] ) * Hillslope )} )}$$

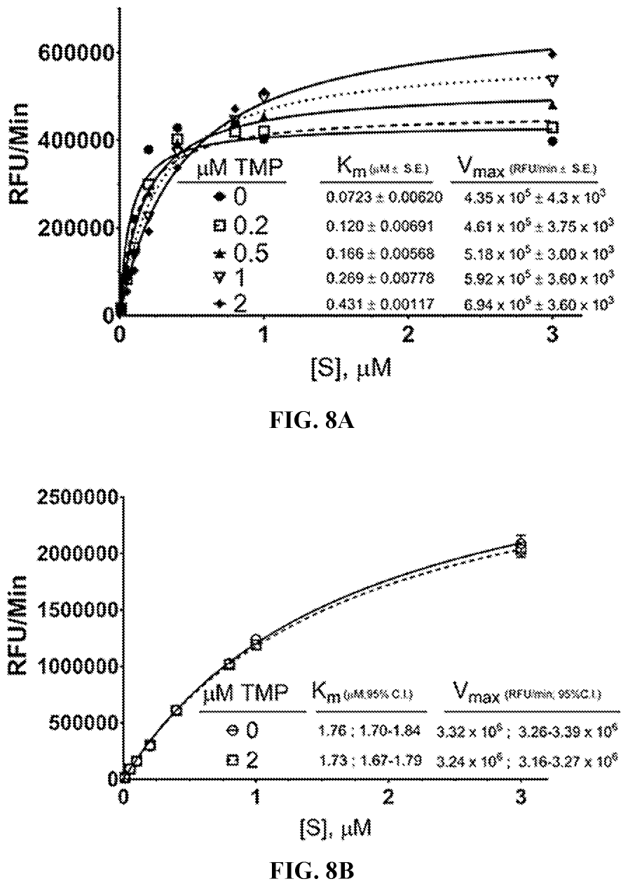

Recifin A inhibitory activity was confirmed in the FRET assay format as shown in FIG. 7. Recifin A inhibited full-length TDP1 enzymatic activity in a concentration-dependent manner with an apparent IC₅₀ of 190 nM. The ability of recific A to inhibit the enzymatic activity of a N-terminal truncated form of TDP1 (Δ147TDP1), in which the regulatory domain had been removed (Huang, et al., Expert Opin. Ther. Pat., 21(9): 1285-92 (2011)) was also evaluated. Only a minimal effect (approximately 20% maximal inhibition) at the highest concentration (1500 nM) was observed. Initial kinetic evaluation of the effect of recifin A on full-length TDP1 activity revealed that sub-micromolar concentrations of recifin A increased the Kₘ for the substrate, broadly defined as an inhibitory characteristic. In addition, a modest increase of the observed Vₘₐₓ value was also detected. This second observation is most often associated with allosteric enzymatic activators (FIG. 8A) (Segel, Wiley: New York, p xxii, 957 p. (1975); Henage, et al., J. Biol. Chem., 281(6): 3408-17 (2006).

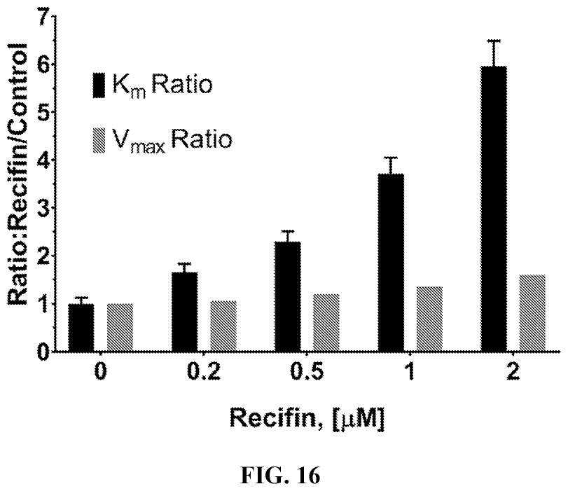

Further analysis of this data revealed that the recifin A-dependent increase in the Kₘ for the substrate is significantly more pronounced (approximately 6-fold higher) than the modest effect on the observed Vₘₐₓ (approximately 1.6-fold higher, FIG. 16), consistent with our initial discovery of this peptide as a TDP1 inhibitor. To further characterize the inhibitory effects of recifin A on TDP1 a FRET based assay was used to determine if recifin A had any effect on the enzymatic activity of Δ147TDP1, lacking the regulatory domain of TDP1. While smaller, Δ147TDP1 retains the substrate binding cleft and dual histidine-lysine-aspartic acid (HKD) motifs responsible for phosphodiesterase catalysis (Davies, et al., Structure, 10(2): 237-48 (2002); Interthal, et al., PNAS, 98(21): 12009-14 (2001)).

As shown in FIG. 8B, recifin A had overlapping 95% confidence intervals for both Kₘ and Vₘₐₓ with the untreated controls, indicating that recifin A does not affect the enzymatic activity of truncated TDP1. This suggests that the binding site for recifin A on TDP1 is outside of the active site region common to both the truncated and full-length forms of TDP1 and is consistent with our results suggesting that recifin A acts as an allosteric modulator of TDP1 enzymatic activity that is binding to the N-terminal TDP1 regulatory domain. Additionally, evaluation of extract of the marine sponge Axinella sp. that yielded recifin A, in an assay to identify inhibitors of the related enzyme tyrosyl-DNA phosphodiesterase II (TDP2), indicated lack of inhibition of TDP2. The lack of activity against this related phosphodiesterase suggests another level of specificity for recifin A against TDP1.

Mechanistically, the recifin A-TDP1 interaction is interesting in that modulators that increase the Kₘ of an enzyme for the substrate are most often characterized as competitive inhibitors. However, the fact that an enzymatically active but truncated form of the protein, with an identical active site, was unaffected by recifin A indicates that the peptide was not directly competing for substrate binding at the active site. Additionally, the observation that recifin A treatment increased the Vₘₐₓ of the enzyme is a general characteristic of an enzymatic activator; further highlighting the novelty of the recifin A-TDP1 interaction and reinforcing the evidence that recifin A does not compete with the phosphotyrosyl-DNA TDP1 substrate. These attributes together in a single interaction are unusual but not without precedent when considering that recifin A is not a small molecule but a complex peptide. There are several classes of enzymes for which a protein-protein interaction is known to change substrate specificity, catalytic efficiency, or both (Pawson, et al., Genes Dev., 14(9): 1027-47 (2000); Haendeler, et al., FEBS Lett, 536(1-3): 180-6 (2003); Moscat, et al., Trends Biochem. Sci., 32(2): 95-100 (2007); Grimsby, et al., Curr. Top Med. Chem., 8(17): 1524-32 (2008)). That recifin A may bind TDP1 allosterically suggests that there may be more to understand about the allosteric regulation of cellular TDP1 activity and that more of the TDP1 protein may be both pharmacologically accessible and therapeutically relevant. It is worth noting that the importance and major topological features present in the first 147 amino acids (deleted from the truncated variant) have not been resolved in a published crystal structure. The few existing publications about this region suggest that it has several known and potential post-translational modification sites (12 predicted according to at least one source), including phosphorylation of serine 81 and SUMOylation of lysine 111, which are important for the regulation of TDP1 intracellular activity (Das, et al., EMBO J., 28(23): 3667-80 (2009); Chiang, et al., Cell Cycle, 9(3): 588-595 (2010); Das, et al., Nucleic Acids Res., 42(7): 4435-49 (2014); Hudson, et al., Nat. Commun., 3: 733 (2012)).

As the data demonstrates, there are substantial enzymatic differences with regard to both Kₘ and Vₘₐₓ of the truncated and full-length TDP1 enzymes.

### Example 5

This example demonstrates the stability of recifin A.

A series of experiments were conducted to determine the stability of recifin A. The results of these studies are summarized as follows:
Recifin A is still active following:
   DTP extract preparation conditions;
   Complete and/or partial drying under nitrogen gas at room temperature;
   Complete and/or partial drying under nitrogen gas at room temperature prior to lyophilization;
   Freezing peptide solutions at −20° C., −80° C., and on dry ice prior to lyophilization;
   Lyophilization; and
   Freezing and thawing processes;
Recifin A is stable in water, PBS pH 7.4, Tris HCl pH 8.0, and solvents methanol, acetonitrile, and DMSO.
Recifin A is stable during standard reversed-phase high performance liquid chromatographic (RP-HPLC) procedures including procedures conducted at room temperature and heated to 40° C., with and without the addition of (0.05%, v/v) TFA (pH approximately equal to 2). Note, RP-HPLC fractions containing recifin A form precipitates upon evaporation of organic solvent when TFA is present.
Recifin A, in its native form, is resistant to digestion with carboxypeptidase Y (1:18 enzyme to target protein ratio by mass, 20 minutes at room temperature).
Recifin A, in its native form, is resistant to digestion with chymotrypsin (1:20 enzyme to target protein ratio by mass, overnight digestion at room temperature).
Recifin A, in its native form, is resistant to digestion with trypsin (1:20 enzyme to target protein ratio by mass, overnight digestion at 37° C.).
Recifin A, in its native form, is resistant to digestion with pyroglutamate aminopeptidase under the following conditions: 2 microgram peptide to 0.2 milliunits enzyme in PBS pH 7.4 buffer, 24 hour digestion at 37 C. Note, when digested under the same conditions in phosphate buffer containing 10 mM DTT, the N-terminal pyroglutamate residue is fully removed.

### Example 6

This example demonstrates that recifin A can be synthetically synthesized providing for generation of analogues.

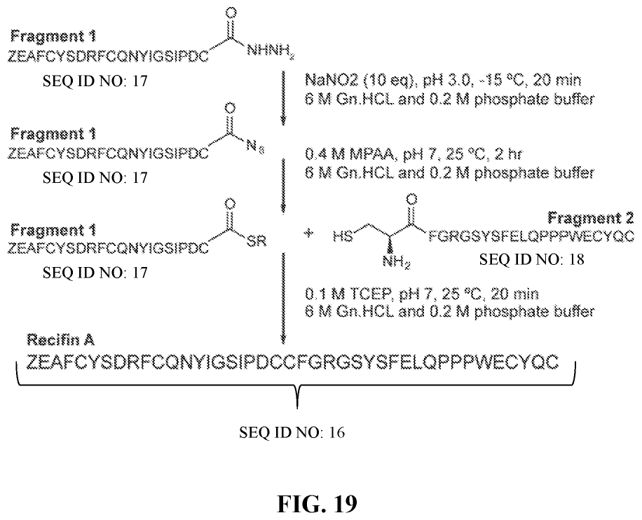

Recombinant production of recifin A in E. coli failed to produce an active protein, and recifin A and analogues thereof could not successfully be assembled in one fragment using Fmoc solid phase peptide synthesis (SPPS). Therefore, a native chemical ligation (NCL) approach using peptide hydrazides was applied to ligate the N- and C-terminal fragments of recifin A and analogues thereof between the 3rd and the 4th cysteine residues (FIG. 19).

The N-terminal peptide hydrazide fragment was synthesized following the protocol established by Zheng et al. Nature Protocols 2013, 8, 2483-2495. Briefly, 2-Cl-(Trt)-Cl (0.5 mmol scale) was washed with DMF three times, DCM three times and DMF three times. The resin was swelled in 50% (v/v) DMF/DCM for 30 mins. After, the solution was drained and 5% (v/v) freshly made NH2NH2 in DMF was added to the resin for hydrazination. The mixture was gently agitated for 30 min at room temperature. The resin was then washed with DMF and DCM three times before repeating incubation with 5% (v/v) freshly made NH2NH2 in DMF for 30 mins. After 30 min the resin was washed with DMF and DCM three times before 5% (v/v) MeOH/DMF was added to the resin and agitated for 10 min to cap unreacted resin. Finally, the resin was washed with DMF three times, DCM three times and DMF three times before manual coupling of the first amino acid. Cysteine(Trt) (4 eq.) was coupled to the hydrazine resin with HBTU (4 eq.) and DIPEA (8 eq.) for 2×1 h. The remainder of fragment 1 for native recifin A and analogies were synthesized using a CS136X synthesizer (CSBio) at 40 degrees C. with Fmoc chemistry using HBTU (0.4 M) and DIPEA (0.8 M) coupling reagents.

For the C-terminal fragment, 2-Cl-(Trt)-Cl (0.25 mmol scale) was swelled in DCM for 30 mins before manual addition of Cys(Trt) (4 eq.) in DCM and DIPEA (8 eq.). A few drops of DMF was added to dissolve the amino acid completely. The amino acid was coupled for 2×1 hr. The remainder of fragment 2 was synthesized using a CS136X synthesizer (CSBio) at 40□ C with Fmoc chemistry using HBTU (0.4 M) and DIPEA (0.8 M) coupling reagents.

Peptides were cleaved from the resin using TFA with DODT, TIPS, and H₂O as scavengers (90:5:2.5:2.5) at room temperature for 2 h. TFA was removed under vacuum and peptide precipitated with ice-cold diethyl ether. The precipitate was filtered and dissolved in 50% acetonitrile containing 0.05% TFA. The remaining diethyl ether was removed under vacuum and the peptide solution lyophilized. Crude peptides were purified by reverse phase-HPLC (RP-HPLC) on a C18 column using a gradient of 0-90% B (Buffer A: 0.05% TFA; Buffer B: 90% ACN/0.045% TFA) in 90 min. Electrospray ionization-mass spectroscopy (ESI-MS) with declustering potential set to 40 was used to confirm the molecular mass of the synthesized peptide fragments using an ABSciex API 2000TM before lyophilization.

Ligation was performed as follows: N-terminal peptide fragment 1-NHNH2 (1 mM) was dissolved in 1 mL of ligation buffer (6 M Gn·HCL, 0.2 M phosphate buffer) and pH was adjusted to ˜3 with 1 M HCL. The peptide solution was cooled in a −15 degrees C. ice/salt bath (12 g NaCl to 50 g of ice) before the addition of NaNO2 (10 eq.). The peptide solution was gently agitated in the ice bath for 20 min to convert the peptide hydrazide to the corresponding azide (N-terminal fragment 1-N3). 0.4 M MPAA was dissolved in 1 mL of ligation buffer and pH was adjusted to 6.8 with 10 M NaOH. C-terminal peptide fragment 2—COOH (1 mM) was dissolved in the 0.4 M MPAA solution and added to the N-terminal fragment 1-N3 solution. The ligation mixture was brought to room temperature and pH was slowly adjusted to 7 using 1 M NaOH. The ligation reaction was left at room temperature for 2 h and monitored using liquid chromatography-mass spectrometry (LC-MS). Upon completion of the reaction, the ligation solution was reduced in 10 mL of 6 M Gn·HCL and 0.1 M TCEP and incubated for 20 mins. After, the ligation solution was diluted tenfold with deionized H2O before being filtered and purified by RP-HPLC on a C18 column using a gradient of 0-90% B in 90 min. ES-MS with declustering potential set to 40 was used to confirm the molecular mass of the ligated peptides before lyophilization.

The full-length ligated peptide was then folded using ammonium bicarbonate buffer with reduced and oxidized glutathione. Pure reduced ligated peptides were dissolved in 0.1 M NH4HCO3 buffer (pH 8) with oxidized (0.5 mM) and reduced (2 mM) glutathione at a concentration of 0.125 mg/mL for 48 h at room temperature. Aliquots of 10 μL were taken at timepoint intervals (0 h, 30 min, 1 h, 2 h, 4 h, 6 h, 8 h, 24 h and 48 h) and quenched in 10 μL 6 M Gn·HCL (pH 3.7). Samples were analyzed by analytical RP-HPLC on a C18 column using a gradient of 5% buffer B for the first 10 min followed by 5-65% B in 65 min. The remaining peptides were oxidized using the above method and were purified by RP-HPLC on a C18 column using a gradient of 0-90% B in 90 min. ESI-MS with declustering potential set to 40 was used to confirm the molecular mass of the oxidized peptides before lyophilization. Analytical RP-HPLC was used to confirm peptide purity.

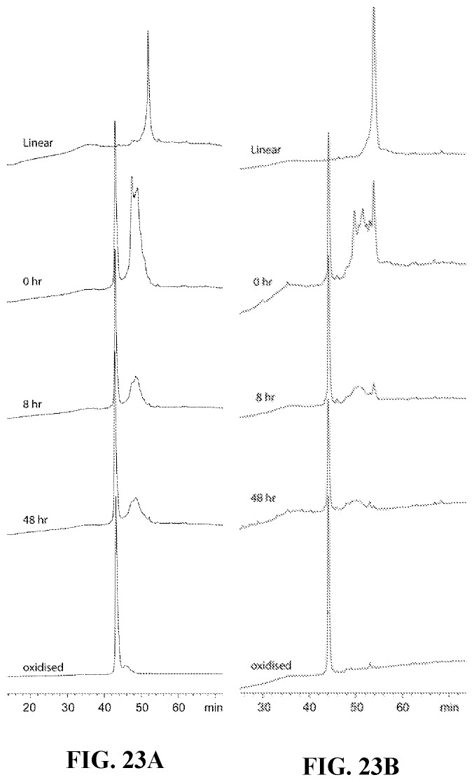

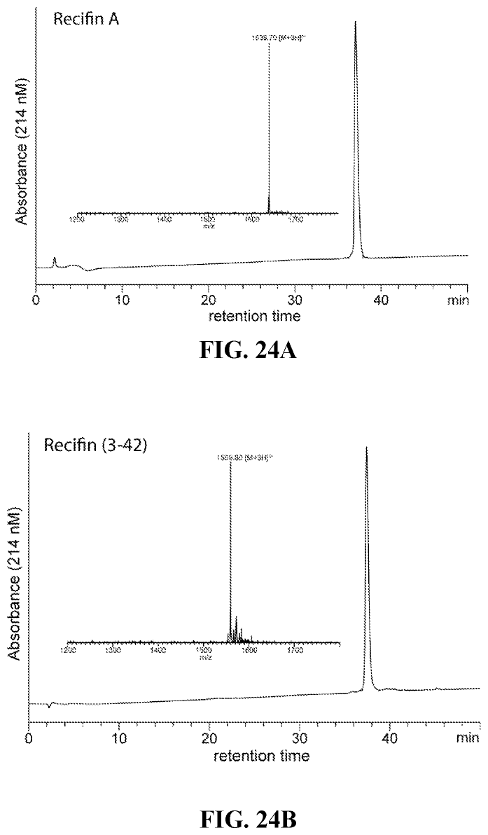

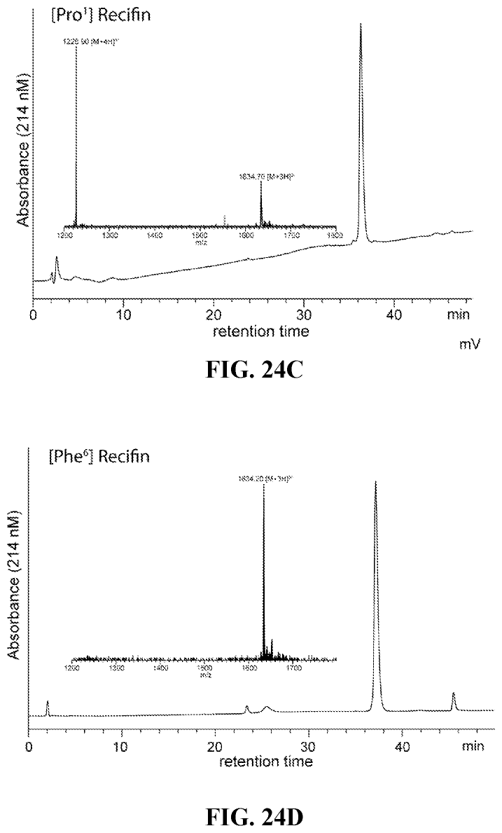

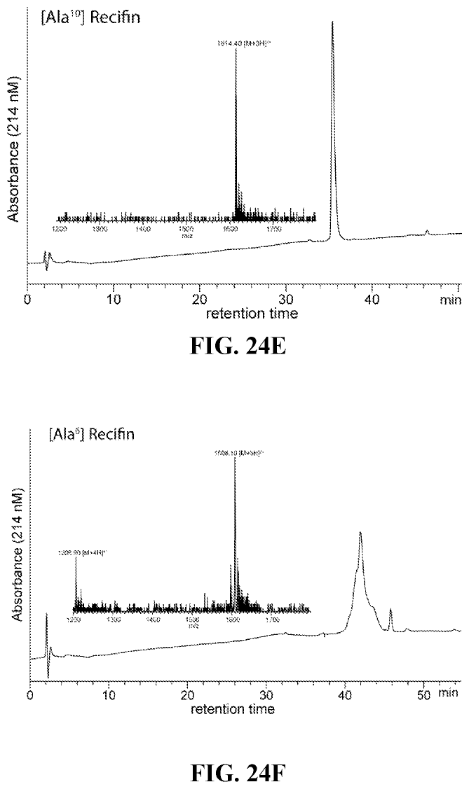

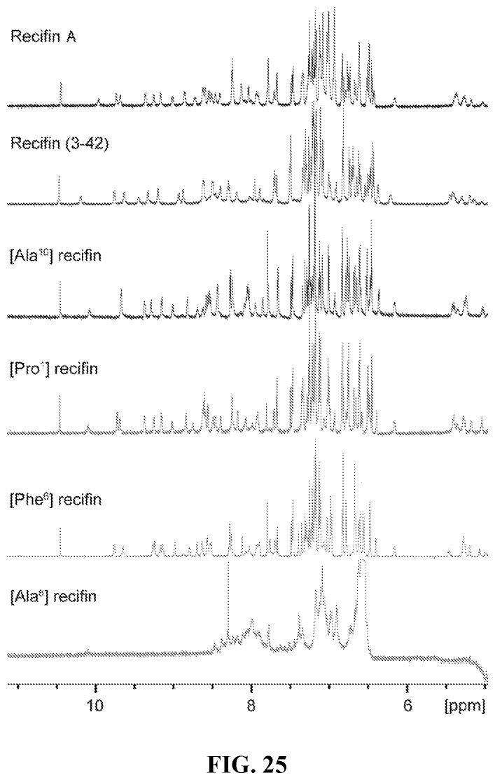

Surprisingly, despite the expected complexity required for correct folding, a single dominant product appeared almost immediately under these conditions (FIGS. 23A-23B). This product was obtained in high purity after HPLC purification (FIGS. 24A-24F) and solution Nuclear Magnetic Resonance (NMR) spectroscopy revealed a well dispersed ¹H NMR spectrum, implying that the peptide adopted an ordered structure in solution (FIG. 25).

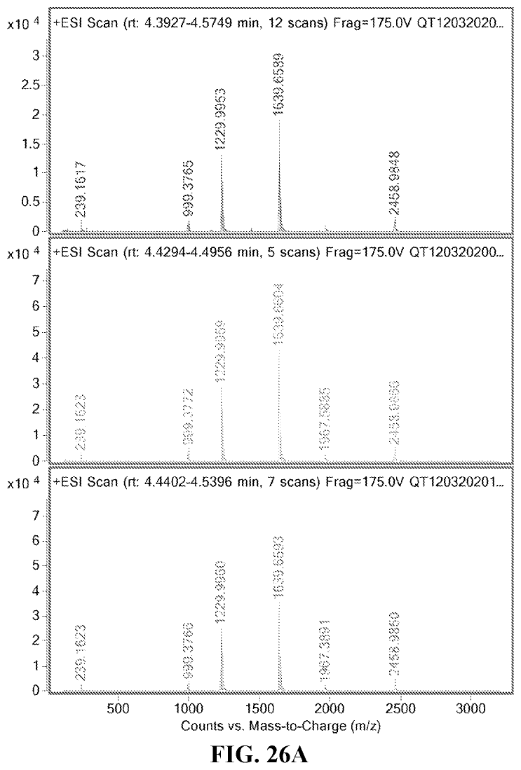

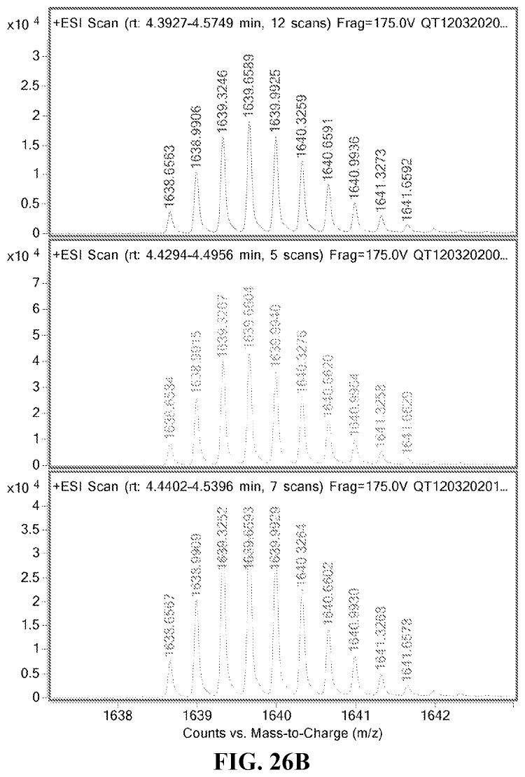

The native isolated recifin A and the synthetic version were compared using LC/MS analysis. Individual analysis of the two peptides found they possessed the same retention time. A co-elution experiment of the two peptides showed no significant difference in retention time or peak shape. Comparing the two recifin A peptide's molecular charge envelope, identical ionization patterns and distribution of charge states were observed, with nearly identical isotopic distribution of [M+3H] ³⁺ (FIGS. 26A-26B). Furthermore, 2D NMR spectra including TOCSY and NOESY were recorded for synthetic recifin A and compared to the data used for structure determination of the native peptide, showing conserved peak patterns and positions (FIG. 18A). The NMR data of recifin A is highly sensitive to minute changes in pH conditions and concentration, making it difficult to replicate conditions perfectly. Consequently, some minor differences in chemical shifts are observed. Taken together these data verify that the synthetic recifin A possesses the same chemical properties as the isolated peptide.

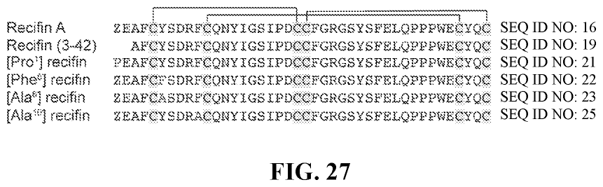

Based on the structural data of the native recifin A peptide, several analogues were synthesized using procedures similar to that provided above to investigate the effects of the mutations on the peptide structure using NMR spectroscopy (Table 8; FIG. 27). Two peptides were designed with mutations at the N-terminus. Recifin 3-42 (SEQ ID NOs: 19) is a truncated version of the native peptide, removing the first two N-terminal residues, pyroglutamic acid and glutamic acid. The [Pro¹] recifin analogue (SEQ ID NO: 21) replaces the N-terminal pyroglutamic acid residue with another five membered ring residue, proline. Two analogues were designed that possessed a mutation of the Tyr6, an important residue that is responsible for further stabilization of the native recifin A peptide. This was replaced with the aromatic residue, phenylalanine ([Phe₆] recifin) (SEQ ID NO: 22), as well as the non-aromatic residue alanine ([Ala⁶] recifin) (SEQ ID NO: 23). A further recifin A analogue that was designed was [Ale¹⁰] recifin (SEQ ID NO: 25), where Phe10 was replaced with Ala. Phenylalanine residues are rarely found on the surface of proteins, unless they are involved in intermolecular interactions. Therefore, it is hypothesized that Phe10 may be a key binding residue and involved in protein-protein interactions between recifin A and the regulatory domain of TDP1.

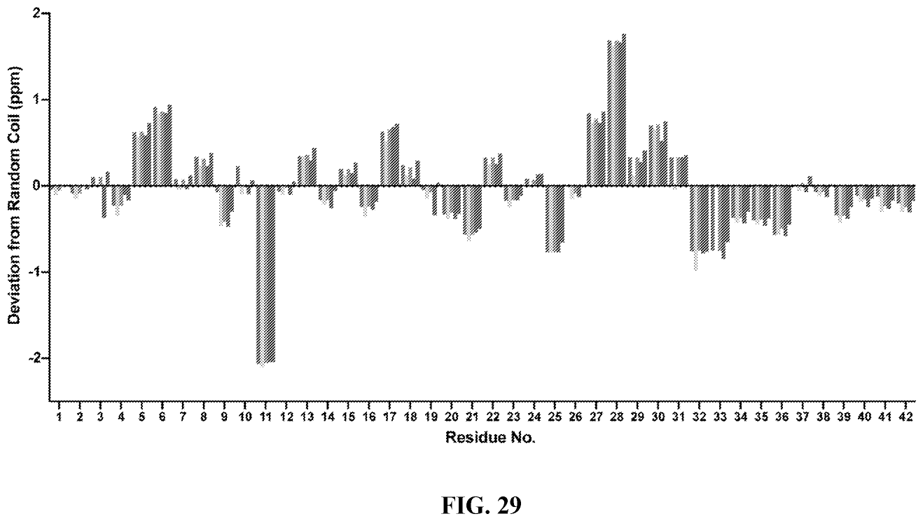

Each recifin analogue was synthesized using NCL and folded with the same conditions as the synthetic recifin A. Most peptide analogues were found to fold into one isomer, which was confirmed by NMR spectroscopy (FIG. 25). [Ala ⁶] recifin 1D NMR spectra appeared broad and the peaks not widely dispersed, indicating a misfolded peptide. The structures of each analogue, except for [Ala ⁶] recifin, were further analyzed by 2D NMR spectroscopy. Secondary Hα chemical shifts revealed that the secondary structural features follow the same trend to that of the native recifin A peptide (FIG. 29). All peptides were shown to possess short β-strands and α-helices/turns, as indicated by significant positive and negative shifts, respectively. [Phe6] recifin was investigated in more detail, given the peptide was able to fold despite a conservative change to the class-defining Tyr6. Calculating a three-dimensional structure of [Phe6] recifin revealed a structure with essentially identical backbone to native recifin A. The key Tyr-lock region observed in the native recifin A structure is maintained in the [Phe6] recifin analogue, despite the loss of a hydrogen bond from the Tyr6 hydroxyl proton to the backbone carbonyl of Glu31. Interestingly two other side chain hydrogen bonds in the region, from Ser27 to Glu8 carbonyl and from Ser29 to Glu31 carbonyl are maintained, as the hydroxyl protons are visible in the spectra, like in native recifin A. Broadening was however observed for the backbone amides of residues 30-33. This indicates that the aromatic ring supplied by a Phe residue is sufficient to maintain the so-called Tyr-lock, although there may be some increased dynamics in the region.

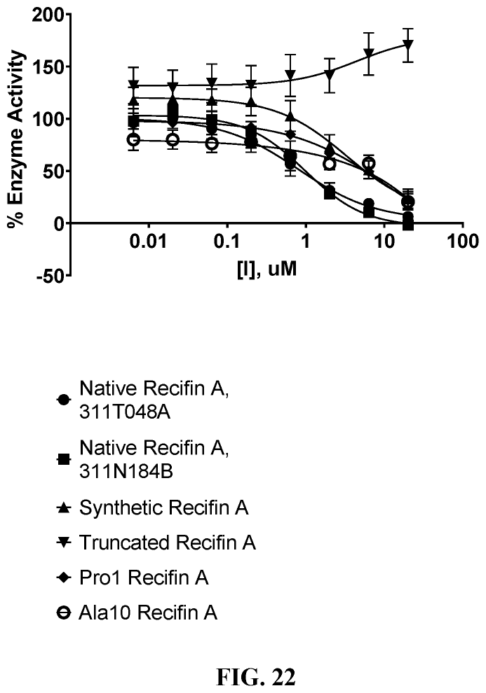

The native recifin A peptide was reported to inhibit full-length TDP1 enzymatic activity in a concentration dependent manner with an IC₅₀ of 0.19 μM. The synthetic recifin A peptide and majority of the analogues were also found to have TDP1 inhibitory activity when using a FRET assay (FIG. 22). Interestingly the truncated analogue recifin 3-42, was found to have no TDP1 inhibitory activity. This suggest that the second residue of the native recifin A, glutamic acid, is important for TDP1 inhibitory activity.

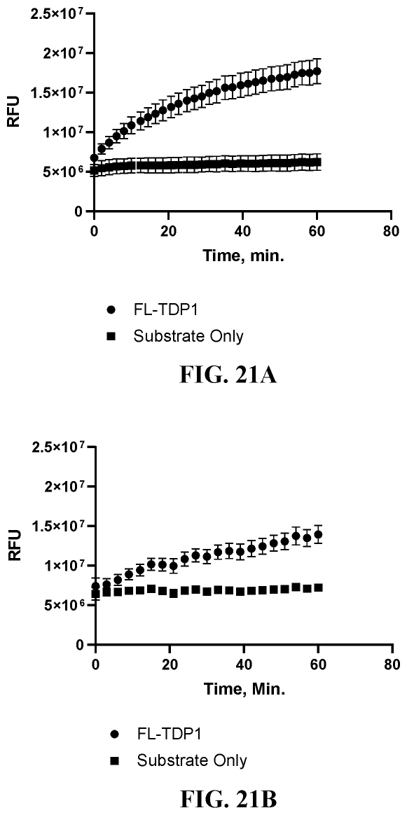

For these FRET assays, TCEP was omitted from the reaction buffer. Peptides were evaluated for FL-TDP1 inhibitory activity using an 8-pt, 10^{0.5} dilution series at a high-test concentration of 20 μM. RXN conditions: 1 nM FL-TDP1, 0.25 μM substrate, T=15 min.; rxn buffer (1XPBS pH 7.4, 80 mM KCl) (-TCEP). See FIGS. 21A and 21B and FIG. 22.

TABLE 7

| PEPTIDE | IC₅₀ μM |
|---|---|
| Native | 1.63 |
| Synthetic | 0.50 |
| Truncated | >10 |
| Pro1 | 0.69 |
| Ala10 | >10 |

TABLE 8
Analogue Sequences

| SEQ | | | |
|---|---|---|---|
| ID | | | |
| NO. | Peptide | Code | Sequence |
| 16 | Recifin | Ref_001 | pGlu-EAFCYSDRFCQNYIGSIPDCCFGRGSYSFELQPPPWECYQC |
| 19 | Recifin (3-42) | Ref_002 | AFCYSDRFCQNYIGSIPDCCFGRGSYSFELQPPPWECYQC |
| 20 | Recifin (5-42) | Ref_003 | (Thz)YSDRFCQNYIGSIPDCCFGRGSYSFELQPPPWECYQC |
| 21 | [Prol]recifin | Ref_004 | PEAFCYSDRFCQNYIGSIPDCCFGRGSYSFELQPPPWECYQC |
| 22 | [Phe6]recifin | Ref_005 | pGlu-EAFCFSDRFCQNYIGSIPDCCFGRGSYSFELQPPPWECYQC |
| 23 | [Ala6]recifin | Ref_006 | pGlu-EAFCASDRFCQNYIGSIPDCCFGRGSYSFELQPPPWECYQC |
| 24 | [Ala9]recifin | Ref_007 | pGlu-EAFCYSDAFCQNYIGSIPDCCFGRGSYSFELQPPPWECYQC |
| 25 | [Ala10]recifin | Ref_008 | pGlu-EAFCYSDRACQNYIGSIPDCCFGRGSYSFELQPPPWECYQC |
| 26 | [Ala35]recifin | Ref_009 | pGlu-EAFCYSDRFCQNYIGSIPDCCFGRGSYSFELQPAPWECYQC |
| 27 | [Arg31]recifin | Ref_010 | pGlu-EAFCYSDRFCQNYIGSIPDCCFGRGSYSFRLQPPPWECYQC |
| 28 | [Arg38]recifin | Ref_011 | pGlu-EAFCYSDRFCQNYIGSIPDCCFGRGSYSFELQPPPWRCYQC |
| 29 | D-recifin | Ref_012 | pGlu-EAFCYSDRFCQNYIGSIPDCCFGRGSYSFELQPPPWECYQC |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A knotted cyclic peptide comprising the amino acid sequence of SEQ ID NO: 11 (CX₁X₂XXXCXXXXXXXXXCCXXXXXXSXXLXXXXXXCXXC), wherein X, X₁, and X₂ can be any amino acid provided that at least one of X₁ and X₂ is tyrosine, phenylalanine, or alanine.

2. The peptide of claim 1, wherein the peptide comprises a four strand anti-parallel β-sheet and two helical turns.

3. The peptide of claim 1, wherein the peptide comprises SEQ ID NO: 7 (CYXXXXCXXXXXXXXXCCXXXXXXSXXLXXXXXXCXXC), wherein X can be any amino acid.

4. The peptide of claim 1, wherein the peptide comprises the amino acid sequence of SEQ ID NO: 1 with an N-terminus truncation of 1, 2, 3, or 4 amino acids.

5. The peptide of claim 1, wherein the peptide does not comprise the amino acid sequence of SEQ ID NO: 1, optionally wherein the peptide comprises about 85-99% sequence identity to SEQ ID NO: 1.

6. The peptide of claim 1, wherein the peptide comprises the amino acid sequence of SEQ ID NO: 7.

7.-10. (canceled)

11. An isolated or purified peptide comprising SEQ ID NO: 1, optionally with 1-6 amino acid substitutions or deletions.

12. A peptide comprising:
(SEQ ID NO: 16)
ZEAFCYSDRFCQNYIGSIPDCCFGRGSYSFELQPPPWECYQC
with one or more of the following modifications:
(a) deletion of residue 1, residues 1 and 2, residues 1-3, or residues 1-4; or substitution Z1P;
(b) Y6F or Y6A;
(c) R9A;
(d) F10A;
(e) E31R;
(f) P35A; and/or
(g) E38R;
or a peptide comprising SEQ ID NO: 16 with one or more of the following modifications:
(a) deletion of residue 1, residues 1 and 2, residues 1-3, or residues 1-4; or substitution Z1P;
(b) Y6F or Y6A; and/or
(c) F10A.

13. The peptide of claim 1, wherein the peptide comprises a disulfide bond network that creates an embedded ring structure.

14. The peptide of claim 1, wherein the peptide is not naturally occurring.

15. The peptide of claim 1, modified with a cell-penetrating peptide sequence.

16. The peptide of claim 1, modified with a cell-penetrating peptide sequence at the N-terminus.

17. The peptide of claim 1, modified with polyethylene glycol.

18. The peptide of claim 1, modified with at least one ethylene glycol at the N-terminus.

19. A pharmaceutical composition comprising (a) the peptide of claim 1 and (b) a pharmaceutically acceptable carrier.

20.-21. (canceled)

22. A method of treating or preventing cancer in a mammal, the method comprising administering to the mammal the peptide of claim 1 in an amount effective to treat or prevent cancer in the mammal.

23. A method of inhibiting the cleavage of phosphodiester bonds by enzyme Tyrosyl-DNA phosphodiesterase 1 (TDP1) in a mammal, the method comprising administering to the mammal the peptide of claim 1 in an amount effective to treat or prevent cancer in the mammal.

24.-26. (canceled)

27. A nucleic acid encoding the peptide of claim 1, optionally in a vector or a cell.

28. A method of preparing the peptide of claim 1, by expressing a nucleic acid encoding the peptide in a host cell, optionally wherein the nucleic acid is in a vector.

29. A method of preparing the peptide of claim 1, comprising (a) synthesizing an N-terminal fragment of the peptide and synthesizing a C-terminal fragment of the peptide, (b) ligating the N-terminal fragment of the peptide to the C-terminal fragment of the peptide to provide the whole peptide, and (c) oxidizing the ligated peptide to induce folding.

30.-32. (canceled)


SEQUENCE LISTING (30 sequences)

SEQ ID NO: 1 - protein, 42 aa, Artificial Sequence
  synthetic
  MISC_FEATURE (1)..(1): wherein Xaa is a pyroglutamic acid
  MISC_FEATURE (5)..(5): wherein Xaa is an iodoacetamide alkylated cysteine
  MISC_FEATURE (11)..(11): wherein Xaa is an iodoacetamide alkylated cysteine
  MISC_FEATURE (21)..(21): wherein Xaa is an iodoacetamide alkylated cysteine
  MISC_FEATURE (22)..(22): wherein Xaa is an N-ethylmaleimide alkylated cysteine
  MISC_FEATURE (39)..(39): wherein Xaa is an iodoacetamide alkylated cysteine
  MISC_FEATURE (42)..(42): wherein Xaa is an N-ethylmaleimide alkylated cysteine
XEAFXYSDRFXQNYIGSIPDXXFGRGSYSFELQPPPWEXYQX

SEQ ID NO: 2 - protein, 41 aa, Artificial Sequence
  synthetic
EAFCYSDRFCQNYIGSIPDCCFGRGSYSFELQPPPWECYQC

SEQ ID NO: 3 - DNA, 14 bp, Artificial Sequence
  synthetic
gatctaaaagactt

SEQ ID NO: 4 - protein, 8 aa, Artificial Sequence
  synthetic
EAFCYSDR

SEQ ID NO: 5 - protein, 16 aa, Artificial Sequence
  synthetic
FCQNYIGSIPDCCFGR

SEQ ID NO: 6 - protein, 15 aa, Artificial Sequence
  synthetic
GSYSFELQPPPQCQC

SEQ ID NO: 7 - protein, 38 aa, Artificial Sequence
  synthetic
  MISC_FEATURE (3)..(3): wherein Xaa can be any naturally occurring amino acid
  MISC_FEATURE (4)..(4): wherein Xaa can be any naturally occurring amino acid
  MISC_FEATURE (5)..(5): wherein Xaa can be any naturally occurring amino acid
  MISC_FEATURE (6)..(6): wherein Xaa can be any naturally occurring amino acid
  MISC_FEATURE (8)..(8): wherein Xaa can be any naturally occurring amino acid
  MISC_FEATURE (9)..(9): wherein Xaa can be any naturally occurring amino acid
  MISC_FEATURE (11)..(11): wherein Xaa can be any naturally occurring amino acid
  MISC_FEATURE (12)..(12): wherein Xaa can be any naturally occurring amino acid
  MISC_FEATURE (13)..(13): wherein Xaa can be any naturally occurring amino acid
  MISC_FEATURE (14)..(14): wherein Xaa can be any naturally occurring amino acid
  MISC_FEATURE (15)..(15): wherein Xaa can be any naturally occurring amino acid
  MISC_FEATURE (16)..(16): wherein Xaa can be any naturally occurring amino acid
  MISC_FEATURE (19)..(19): wherein Xaa can be any naturally occurring amino acid
  MISC_FEATURE (20)..(20): wherein Xaa can be any naturally occurring amino acid
  MISC_FEATURE (21)..(21): wherein Xaa can be any naturally occurring amino acid
  MISC_FEATURE (22)..(22): wherein Xaa can be any naturally occurring amino acid
  MISC_FEATURE (23)..(23): wherein Xaa can be any naturally occurring amino acid
  MISC_FEATURE (24)..(24): wherein Xaa can be any naturally occurring amino acid
  MISC_FEATURE (26)..(26): wherein Xaa can be any naturally occurring amino acid
  MISC_FEATURE (27)..(27): wherein Xaa can be any naturally occurring amino acid
  MISC_FEATURE (29)..(29): wherein Xaa can be any naturally occurring amino acid
  MISC_FEATURE (30)..(30): wherein Xaa can be any naturally occurring amino acid
  MISC_FEATURE (31)..(31): wherein Xaa can be any naturally occurring amino acid
  MISC_FEATURE (32)..(32): wherein Xaa can be any naturally occurring amino acid
  MISC_FEATURE (33)..(33): wherein Xaa can be any naturally occurring amino acid
  MISC_FEATURE (34)..(34): wherein Xaa can be any naturally occurring amino acid
  MISC_FEATURE (36)..(36): wherein Xaa can be any naturally occurring amino acid
  MISC_FEATURE (37)..(37): wherein Xaa can be any naturally occurring amino acid
CYXXXXCXXYXXXXXXCCXXXXXXSXXLXXXXXXCXXC

SEQ ID NO: 8 - protein, 38 aa, Artificial Sequence
  synthetic
  MISC_FEATURE (4)..(4): wherein Xaa can be any naturally occurring amino acid
  MISC_FEATURE (5)..(5): wherein Xaa can be any naturally occurring amino acid
  MISC_FEATURE (6)..(6): wherein Xaa can be any naturally occurring amino acid
  MISC_FEATURE (8)..(8): wherein Xaa can be any naturally occurring amino acid
  MISC_FEATURE (9)..(9): wherein Xaa can be any naturally occurring amino acid
  MISC_FEATURE (11)..(11): wherein Xaa can be any naturally occurring amino acid
  MISC_FEATURE (14)..(14): wherein Xaa can be any naturally occurring amino acid
  MISC_FEATURE (15)..(15): wherein Xaa can be any naturally occurring amino acid
  MISC_FEATURE (16)..(16): wherein Xaa can be any naturally occurring amino acid
  MISC_FEATURE (19)..(19): wherein Xaa can be any naturally occurring amino acid
  MISC_FEATURE (20)..(20): wherein Xaa can be any naturally occurring amino acid
  MISC_FEATURE (21)..(21): wherein Xaa can be any naturally occurring amino acid
  MISC_FEATURE (22)..(22): wherein Xaa can be any naturally occurring amino acid
  MISC_FEATURE (26)..(26): wherein Xaa can be any naturally occurring amino acid
  MISC_FEATURE (29)..(29): wherein Xaa can be any naturally occurring amino acid
  MISC_FEATURE (30)..(30): wherein Xaa can be any naturally occurring amino acid
  MISC_FEATURE (31)..(31): wherein Xaa can be any naturally occurring amino acid
  MISC_FEATURE (34)..(34): wherein Xaa can be any naturally occurring amino acid
  MISC_FEATURE (37)..(37): wherein Xaa can be any naturally occurring amino acid
CYSXXXCXXYXGSXXXCCXXXXSYSXELXXXPWXCYXC

SEQ ID NO: 9 - protein, 38 aa, Artificial Sequence
  synthetic
  MISC_FEATURE (3)..(3): Xaa can be any naturally occurring amino acid
  MISC_FEATURE (4)..(4): Xaa can be any naturally occurring amino acid
  MISC_FEATURE (8)..(8): Xaa can be any naturally occurring amino acid
  MISC_FEATURE (9)..(9): Xaa can be any naturally occurring amino acid
  MISC_FEATURE (11)..(11): Xaa can be any naturally occurring amino acid
  MISC_FEATURE (12)..(12): Xaa can be any naturally occurring amino acid
  MISC_FEATURE (13)..(13): Xaa can be any naturally occurring amino acid
  MISC_FEATURE (14)..(14): Xaa can be any naturally occurring amino acid
  MISC_FEATURE (15)..(15): Xaa can be any naturally occurring amino acid
  MISC_FEATURE (16)..(16): Xaa can be any naturally occurring amino acid
  MISC_FEATURE (19)..(19): Xaa can be any naturally occurring amino acid
  MISC_FEATURE (20)..(20): Xaa can be any naturally occurring amino acid
  MISC_FEATURE (22)..(22): Xaa can be any naturally occurring amino acid
  MISC_FEATURE (23)..(23): Xaa can be any naturally occurring amino acid
  MISC_FEATURE (24)..(24): Xaa can be any naturally occurring amino acid
  MISC_FEATURE (26)..(26): Xaa can be any naturally occurring amino acid
  MISC_FEATURE (27)..(27): Xaa can be any naturally occurring amino acid
  MISC_FEATURE (29)..(29): Xaa can be any naturally occurring amino acid
  MISC_FEATURE (30)..(30): Xaa can be any naturally occurring amino acid
  MISC_FEATURE (31)..(31): Xaa can be any naturally occurring amino acid
  MISC_FEATURE (32)..(32): Xaa can be any naturally occurring amino acid
  MISC_FEATURE (34)..(34): Xaa can be any naturally occurring amino acid
  MISC_FEATURE (36)..(36): Xaa can be any naturally occurring amino acid
  MISC_FEATURE (37)..(37): Xaa can be any naturally occurring amino acid
CYXXRFCXXYXXXXXXCCXXRXXXSXXLXXXXWXCXXC

SEQ ID NO: 10 - protein, 41 aa, Artificial Sequence; identical to SEQ ID NO: 2
  synthetic

SEQ ID NO: 11 - protein, 38 aa, Artificial Sequence
  synthetic
  MISC_FEATURE (2)..(3): Xaa is any amino acid, but at least one of residue 2 and residue 3 is tyrosine
  MISC_FEATURE (2)..(2): wherein residue 2 is any amino acid if residue 3 is tyrosine
  MISC_FEATURE (3)..(3): wherein residue 3 is any amino acid if residue 2 is tyrosine
  MISC_FEATURE (4)..(4): Xaa can be any naturally occurring amino acid
  MISC_FEATURE (5)..(5): Xaa can be any naturally occurring amino acid
  MISC_FEATURE (6)..(6): Xaa can be any naturally occurring amino acid
  MISC_FEATURE (8)..(8): Xaa can be any naturally occurring amino acid
  MISC_FEATURE (9)..(9): Xaa can be any naturally occurring amino acid
  MISC_FEATURE (10)..(10): Xaa can be any naturally occurring amino acid
  MISC_FEATURE (11)..(11): Xaa can be any naturally occurring amino acid
  MISC_FEATURE (12)..(12): Xaa can be any naturally occurring amino acid
  MISC_FEATURE (13)..(13): Xaa can be any naturally occurring amino acid
  MISC_FEATURE (14)..(14): Xaa can be any naturally occurring amino acid
  MISC_FEATURE (15)..(15): Xaa can be any naturally occurring amino acid
  MISC_FEATURE (16)..(16): Xaa can be any naturally occurring amino acid
  MISC_FEATURE (19)..(19): Xaa can be any naturally occurring amino acid
  MISC_FEATURE (20)..(20): Xaa can be any naturally occurring amino acid
  MISC_FEATURE (21)..(21): Xaa can be any naturally occurring amino acid
  MISC_FEATURE (22)..(22): Xaa can be any naturally occurring amino acid
  MISC_FEATURE (23)..(23): Xaa can be any naturally occurring amino acid
  MISC_FEATURE (24)..(24): Xaa can be any naturally occurring amino acid
  MISC_FEATURE (26)..(26): Xaa can be any naturally occurring amino acid
  MISC_FEATURE (27)..(27): Xaa can be any naturally occurring amino acid
  MISC_FEATURE (29)..(29): Xaa can be any naturally occurring amino acid
  MISC_FEATURE (30)..(30): Xaa can be any naturally occurring amino acid
  MISC_FEATURE (31)..(31): Xaa can be any naturally occurring amino acid
  MISC_FEATURE (32)..(32): Xaa can be any naturally occurring amino acid
  MISC_FEATURE (33)..(33): Xaa can be any naturally occurring amino acid
  MISC_FEATURE (34)..(34): Xaa can be any naturally occurring amino acid
  MISC_FEATURE (36)..(36): Xaa can be any naturally occurring amino acid
  MISC_FEATURE (37)..(37): Xaa can be any naturally occurring amino acid
CXXXXXCXXXXXXXXXCCXXXXXXSXXLXXXXXXCXXC

SEQ ID NO: 12 - protein, 38 aa, Artificial Sequence
  synthetic
  MISC_FEATURE (4)..(4): Xaa can be any naturally occurring amino acid
  MISC_FEATURE (8)..(8): Xaa can be any naturally occurring amino acid
  MISC_FEATURE (9)..(9): Xaa can be any naturally occurring amino acid
  MISC_FEATURE (11)..(11): Xaa can be any naturally occurring amino acid
  MISC_FEATURE (14)..(14): Xaa can be any naturally occurring amino acid
  MISC_FEATURE (15)..(15): Xaa can be any naturally occurring amino acid
  MISC_FEATURE (16)..(16): Xaa can be any naturally occurring amino acid
  MISC_FEATURE (19)..(19): Xaa can be any naturally occurring amino acid
  MISC_FEATURE (20)..(20): Xaa can be any naturally occurring amino acid
  MISC_FEATURE (22)..(22): Xaa can be any naturally occurring amino acid
  MISC_FEATURE (26)..(26): Xaa can be any naturally occurring amino acid
  MISC_FEATURE (29)..(29): Xaa can be any naturally occurring amino acid
  MISC_FEATURE (30)..(30): Xaa can be any naturally occurring amino acid
  MISC_FEATURE (31)..(31): Xaa can be any naturally occurring amino acid
  MISC_FEATURE (34)..(34): Xaa can be any naturally occurring amino acid
  MISC_FEATURE (37)..(37): Xaa can be any naturally occurring amino acid
CYSXRFCXXYXGSXXXCCXXRXSYSXELXXXPWXCYXC

SEQ ID NO: 13 - protein, 40 aa, Artificial Sequence
  synthetic
AFCYSDRFCQNYIGSIPDCCFGRGSYSFELQPPPWECYQC

SEQ ID NO: 14 - protein, 39 aa, Artificial Sequence
  synthetic
FCYSDRFCQNYIGSIPDCCFGRGSYSFELQPPPWECYQC

SEQ ID NO: 15 - protein, 38 aa, Artificial Sequence
  synthetic
CYSDRFCQNYIGSIPDCCFGRGSYSFELQPPPWECYQC

SEQ ID NO: 16 - protein, 42 aa, Artificial Sequence
  synthetic
ZEAFCYSDRFCQNYIGSIPDCCFGRGSYSFELQPPPWECYQC

SEQ ID NO: 17 - protein, 21 aa, Artificial Sequence
  synthetic
ZEAFCYSDRFCQNYIGSIPDC

SEQ ID NO: 18 - protein, 20 aa, Artificial Sequence
  synthetic
FGRGSYSFELQPPPWECYQC

SEQ ID NO: 19 - protein, 40 aa, Artificial Sequence; identical to SEQ ID NO: 13
  synthetic

SEQ ID NO: 20 - protein, 38 aa, Artificial Sequence
  synthetic
  MISC_FEATURE (1)..(1): wherein Xaa is (4R)-1,3-thiazolidine-4- carboxylic acid
XYSDRFCQNYIGSIPDCCFGRGSYSFELQPPPWECYQC

SEQ ID NO: 21 - protein, 42 aa, Artificial Sequence
  synthetic
PEAFCYSDRFCQNYIGSIPDCCFGRGSYSFELQPPPWECYQC

SEQ ID NO: 22 - protein, 42 aa, Artificial Sequence
  synthetic
  MISC_FEATURE (1)..(1): wherein Xaa is a pyroglutamic acid
XEAFCFSDRFCQNYIGSIPDCCFGRGSYSFELQPPPWECYQC

SEQ ID NO: 23 - protein, 42 aa, Artificial Sequence
  synthetic
  MISC_FEATURE (1)..(1): wherein Xaa is a pyroglutamic acid
XEAFCASDRFCQNYIGSIPDCCFGRGSYSFELQPPPWECYQC

SEQ ID NO: 24 - protein, 42 aa, Artificial Sequence
  synthetic
  MISC_FEATURE (1)..(1): wherein Xaa is a pyroglutamic acid
XEAFCYSDAFCQNYIGSIPDCCFGRGSYSFELQPPPWECYQC

SEQ ID NO: 25 - protein, 42 aa, Artificial Sequence
  synthetic
  MISC_FEATURE (1)..(1): wherein Xaa is a pyroglutamic acid
XEAFCYSDRACQNYIGSIPDCCFGRGSYSFELQPPPWECYQC

SEQ ID NO: 26 - protein, 42 aa, Artificial Sequence
  synthetic
  MISC_FEATURE (1)..(1): wherein Xaa is a pyroglutamic acid
XEAFCYSDRFCQNYIGSIPDCCFGRGSYSFELQPAPWECYQC

SEQ ID NO: 27 - protein, 42 aa, Artificial Sequence
  synthetic
  MISC_FEATURE (1)..(1): wherein Xaa is a pyroglutamic acid
XEAFCYSDRFCQNYIGSIPDCCFGRGSYSFRLQPPPWECYQC

SEQ ID NO: 28 - protein, 42 aa, Artificial Sequence
  synthetic
  MISC_FEATURE (1)..(1): wherein Xaa is a pyroglutamic acid
XEAFCYSDRFCQNYIGSIPDCCFGRGSYSFELQPPPWRCYQC

SEQ ID NO: 29 - protein, 42 aa, Artificial Sequence
  synthetic
  MISC_FEATURE (1)..(1): wherein Xaa is a pyroglutamic acid
XEAFCYSDRFCQNYIGSIPDCCFGRGSYSFELQPPPWECYQC

SEQ ID NO: 30 - protein, 40 aa, Artificial Sequence
  synthetic
GVFCYSDRFCQNPIDNFDCCFSRGSYSFVPQPTPWDCFQC